(19) United States
(12) Patent Application Publication
(10) Pub. No.: US 2025/0197790 A1
(43) Pub. Date: Jun. 19, 2025

(54) COATED ARTICLE FOR CULTURING PRIMARY CELLS AND STEM CELLS AND METHOD FOR PREPARING THE SAME

(71) Applicant: denovoMATRIX GmbH, Dresden (DE)

(72) Inventors: Thomas HENDEL, Dresden (DE); Sandra SEGELETZ, Dresden (DE); Kristina THAMM, Dresden (DE); Richard WETZEL, Dresden (DE)

(21) Appl. No.: 18/849,908

(22) PCT Filed: Mar. 30, 2023
(86) PCT No.: PCT/EP2023/058315

§ 371 Date: Sep. 23, 2024

(30) Foreign Application Priority Data
Mar. 30, 2022 (EP) 22165448.6

(51) Int. Cl. C12M 1/12; C12M 1/00; C12N 5/00

(52) U.S. Cl. CPC C12M 25/14; C12M 23/20; C12N 5/0068; C12N 2513/00; C12N 2533/40; C12N 2533/50; C12N 2533/70

(57) ABSTRACT

The invention relates to a coated article for culturing primary cells and stem cells, said coated article comprising a cell culture scaffold material with a non-covalent coating, said non-covalent coating comprises or consists of at least one layer consisting of at least one negatively charged polysaccharide and a layer consisting of a positively charged conjugate. The invention further relates to a method of preparing said coated article and its use in regenerative medicine, gene and cell therapy, aesthetic medicine, or cellular agriculture.

### FIELD OF THE INVENTION

The invention relates to a coated article for culturing primary cells and stem cells, said coated article comprising a cell culture scaffold material with a non-covalent coating, said non-covalent coating comprises or consists of a multilayer coating comprising at least one layer consisting of at least one negatively charged polysaccharide (NCP) and a layer consisting of a positively charged conjugate. The invention further relates to a method of preparing said coated article and its use in regenerative medicine, gene and cell therapy, aesthetic medicine, and in cellular agriculture.

### BACKGROUND OF THE INVENTION

The industrial-scale cultivation of human cells for medicinal products and of animal derived cells for cellular agriculture is an outstanding biological and logistic problem. Research-scale cell cultivation is currently realized by growing cells on 2-dimensional (2D) cell culture plates/stacks. Industrial-scale cell cultivation requires more cost-effective culture formats with increased process control and reduced human interactions such as bioreactors. Within bioreactors cells are cultured in 3-dimensional (3D) manner as aggregates or on scaffolds such as microcarriers, hollow fibers, or sponge-like materials. A variety of materials have been used to produce these scaffolds, such as polystyrene, dextran, poly-lactic-glycolic acid (PLGA), alginate, collagen, gelatin, silk in both bulk and porous structures¹⁻³. Applying such materials for anchorage-dependent cells requires further modifications. Anchorage-dependent cells like embryonic, adult or induced stem cells, progenitor cells, and terminally differentiated primary cells strictly rely on adhesion ligands derived from their natural extracellular matrix (ECM) for their growth and survival. Therefore, 2D and 3D surfaces are routinely coated with substrates that aim to recreate the essential cues of the cellular microenvironment necessary for their growth. There are a variety of biological, regulatory, and economic constraints on the nature of those coating materials, their manufacturing, their production, and on the coating procedure itself for their use in regenerative medicine, gene and cell therapy, aesthetic medicine, or cellular agriculture.

Regenerative and aesthetic medicine defines a medical treatment of any type of regeneration, supplementation, or replacement of cells, organs or tissues with cells, engineered cell-tissue or cultured organs or similar living therapeutics. Cellular agriculture is defined as production of agriculture products from cell cultures derived from wild or domesticated animals.

In addition, there is a challenge of upscaling cell procedures from laboratory scale to industrial/manufacturing scale. Laboratory scale of cell culture in regenerative medicine and cellular agriculture are usually conducted on 2D surfaces (<150 cm²). In transition from 2D processes to 3D processes it is necessary to change most supporting procedures including ECM coatings, which introduces additional uncertainty and costs, and which is not always possible. Therefore, most of the current coating materials are limited in their application to industrial-scale cultivation of mammalian cells. The present invention provides a universally transferable coating and coating method, which is applicable to laboratory and industrial scale alike.

One of the most frequently applied scaffolds for industrial cell manufacturing are microcarriers. Microcarriers provide large surface areas to support the growth and attachment of cells and can be implemented in stirred-tank bioreactors at industrial scale. Therefore, the use of microcarriers has been established since the 1980's in biopharmaceutical industry to produce vaccines and recombinant proteins by mammalian cell lines. Growth and maintenance of cells in an industrial-scale bioreactor is more efficient and economically more feasible than growing cells on 2D surfaces and allows constant monitoring of the culture conditions and streamlined processes. The culture of anchorage-dependent stem cells, progenitor cells, and different primary cells requires specific adhesion ligands and cannot be grown on untreated microcarriers. Microcarriers have been chemically modified⁴ to introduce functional charged groups for supporting cell adhesion. However, for most cell types either the initial adhesion is not sufficient to enable cell survival and initiate cell growth or cell adhesion is too strong and cell harvest is limited on such microcarriers. Therefore, microcarriers are modified with ECM substrates or proteins or peptides to support the adhesion of those cell types.

Since the first application of cancer tissue extracts in 1986 by Kleinman and colleagues for cell culture, multiple ECM substrates have been developed with Matrigel being the most used in primary and stem cell cultures^{5,6}. Most of these substrates are not eligible for regenerative medicine, aesthetic medicine, or cellular agriculture due to their poor chemical definition and animal origin. However, proteins and peptides produced in vitro can be used in such applications. Coatings based on these components aim to recreate the minimal essential cues of the cellular microenvironment necessary for cell growth. ECM-proteins like fibronectin, vitronectin, and laminin are self-assembling and coat suitable surfaces by charge-driven adsorption⁷. This approach is simple and avoids chemical crosslinking for stable immobilization of proteins on the surfaces which is beneficial for industrial processes.

Culture coatings made of full-length proteins are challenging to implement into industrial processes. Proteins are prone to denature and degenerate under ambient storage conditions and thus, tend to be complex and demanding in terms of storage (short shelf-life, low-temperature storage) or require in situ coating procedures. To overcome these problems, peptide-based coatings present an alternative solution as they are fully chemically defined, easy to scale in production, and more robust for storage.

Peptide coated substrates are often applied to solely support the integrin-binding of the cells⁸. This minimal approach does not reflect the full complexity and functionality of the natural ECM⁹. The ECM also contains polysaccharides that provide mechanical support, serve as an aqueous reservoir, and aid in cell proliferation and function in addition to cell binding¹⁰⁻¹². To address cell type-specific needs, it is important to use coating compositions that fulfill industry standards as well as the cell's biological requirements.

The use of cultured cells for/in humans also poses regulatory requirements on cell culture coatings. ECM extracts, as well as recombinant proteins, exhibit batch-to-batch variability due to their nature, which hinders reliable outcomes. Potential impurities and viral or prion contamination represent an additional risk and inhibit their application in the context of e.g. regenerative medicine and cell therapies. Alternative synthetic coatings often rely on covalent surface modifications that change the properties of the microcarrier raw material, which requires renewal of regulatory approval of a cell therapy process. Finally, like other ancillary materials for cell culture, coating materials need to be obtainable at pharma- or food-grade quality and at bulk quantity.

To address the plethora of demands on the nature and application of coatings culture of primary cells and stem cells, denovoMATRIX has previously invented a technology using polysaccharides and polymers conjugated with specific peptide sequences to form a non-covalent hydrogel by a coacervation-based method¹³. The formation of the hydrogel is driven by self-assembly and phase separation in aqueous solution (coacervation) followed by gravity-based deposition. This approach has been demonstrated to enable the formation of stable bulk hydrogels for cell culture, cell encapsulation and bio-engineering tasks^{14,15}.

Based on this approach, a method has been developed to facilitate surface coating applications of cell culture articles. The coating application was previously reported to support serum-free culture of mesenchymal stromal cells useable for regenerative medicine^{16,17}. However, the gravity dependency of this conventional method is limiting its application to planar surfaces (2D surfaces).

Further, the application of this conventional coating procedure at industrial scale is limited by i) the sedimentation time of the formed coacervates from solution to the surfaces and ii) the high consumption of the coating materials used. Layers prepared with this conventional approach have usually a thickness in the μm range, typically about 10 μm.

A layer-by-layer (LbL) based coating method can overcome these limitations. Thomas et al. have shown that an LbL method can be applied using the polysaccharide heparin and starPEG-peptide-conjugates to create surface coatings with a linear growth pattern¹⁸. The reported method overcomes the drawbacks of the coacervation method by focusing on molecular charge interaction in a proof-of-principle study. However, the reported method has limited potential for application in cell culture industry. One aspect is the necessity of covalent coupling of the founding layer (in this example heparin has been used) to a glass support by silanization chemistry. This restricts its industrial application in two ways. First, covalent coupling in wet chemistry introduces an unfavorable complexity and additional time constraints, which are to be avoided for industrial processes. Second and most important, the method is restricted to SiO2-based materials like glass whereas most cell culture scaffolds are based on natural or synthetic polymers like dextran and polystyrene. In another aspect, the report demonstrates the principal suitability of the LbL approach to create a coating for cell culture. However, the employed cell model of fibroblast does not sufficiently demonstrate the eligibility of the reported formulation for more demanding cell types like primary cells and stem cells.

Purpose of the present invention has been to overcome the obstacles of the prior art and to provide a universal coating for cell-culture-relevant scaffold materials of different types, in particular 3D-scaffolds, preferably microcarriers for culture of mammalian cells, in particular primary cells, stem cells and cells derived thereof.

### DESCRIPTION OF THE INVENTION

The present invention addresses the limitations of the prior art by focusing on a non-covalent purely charged-based coating that can be modified towards the various surface properties of cell culture scaffold materials. The invention further addresses the requirements of adherent primary cells and stem cells in an effective manner by integration of specific peptide sequences and optimized coating conditions.

The previously described limitations, that only 2D surfaces can be coated, are solved with this invention by applying a stepwise coating with several components in an LbL method wherein the coating is driven by adsorption und charge interaction.

In a preferred embodiment, the invention provides a coated article for culturing primary cells and stem cells, said coated article comprising a cell culture scaffold material with a non-covalent coating, said non-covalent coating comprises or consists of at least one layer consisting of at least one negatively charged polysaccharide (NCP), a layer consisting of a positively charged conjugate and optionally a layer comprising or consisting of a charge-inducing component.

As it is known to the person skilled in the art, a non-covalent interaction differs from a covalent bond in that it does not form molecular orbitals, but rather involves more dispersed variations of electromagnetic interactions between molecules or within a molecule. Commonly, non-covalent interactions are considered weaker than covalent bonds, i.e. non-covalent interactions have lower dissociation energies. Typical non-covalent interactions include electrostatic interactions such as polar interactions, hydrogen bonding, halogen bonding, van der Waals forces including dipole-dipole, and induced dipole interactions as well as London dispersion forces, and π effects like π-π interactions, cation-π and anion-π interactions, as well as hydrophobic effects.

Said cell culture scaffold material usually comprises a negatively charged surface, a neutral (i.e. uncharged) surface or a positively charged surface.

The charge of the surface of the cell culture scaffold material can be measured with conventional methods known to the person skilled in the art. A suitable method for determining the surface charge of the cell culture scaffold material is measuring the zeta potential at the surface when the cell culture scaffold material is selected from spherical 3D-particles with a particle size below 100 μm. The surface coating can be monitored by fluorescence microcopy when the cell culture scaffold material is a spherical 3D-particle with a particle size larger than 100 μm, a non-spherical 3D-particle or network or is a 2D-material, e.g. as described in the examples of the invention. These methods are also applicable when the cell culture scaffold material is porous.

Zeta potential is a scientific term for the electrokinetic potential on the surface of a dispersed colloid. Around every colloidal particle dispersed in a solution an electrical double layer is formed due to charges on the particle surface affecting the distribution of ions at the interface with the surrounding solution. Brownian motion of the particle results in shearing forces which remove loosely bound ions while the inner firmly bound ions remain with the particle. The resulting electrical potential at the interface is defined as zeta potential and is a proportional measure for the otherwise unmeasurable net surface charge of the particle.

In the practical sense of the present invention, the surface of the cell culture scaffold material comprised in the coated article of the invention is negatively charged when the value of zeta potential is below −10 mV, for example between −250 mV and −10 mV. The surface of the cell culture scaffold material comprised in the coated article of the invention is neutral (i.e. uncharged), when the value of zeta potential is between −10 mV and 10 mV. The surface of the cell culture scaffold material comprised in the coated article of the invention is positively charged, when the value of zeta potential is higher than 10 mV, for example between 10 mV and 250 mV.

The finally coated article, i.e. after application of certain coating layers, shows a surface charge in the range between −50 mV and 50 mV, preferably between −30 mV and 30 mV, more preferably between −10 mV and 10 mV, most preferably between −3 mV and 3 mV. A suitable method for determining the surface charge of the finally coated article is measuring its zeta potential. The method is described in the examples of the invention.

Another possibility to estimate the charge of a scaffold is to label it with charged biomaterials and examine the scaffold with fluorescence microscopy. While the NCP is negatively charged, the conjugate exhibits a positive charge. By attaching a fluorescent label to these polymers, they can be detected on the surface of any scaffold. Due to the charged nature of the coating materials used, the charge of the scaffold surface can be deduced. Fluorescent microscopy does not only allow for qualitative statements on the charge of a given scaffold. The determined intensity is a measure for the amount of biomaterial deposited on the scaffold and likewise, can be used indirectly to quantify the charge on the scaffold. To exemplify, scaffolds are coated with NCP and conjugate using the LbL technique of the invention. For fluorescent imaging, the conjugate contains a portion of a Cy5-labeled conjugate and the NCP contains a portion of a FITC- or TRITC-labeled NCP. With this setup, coated scaffolds can be imaged using fluorescence microscopy and thus, the coating efficiency is monitored, and the charge of the scaffold is deduced.

A positively charged scaffold is the most preferred base material for the LbL coating approach of the invention. It enables an effective deposition of NCPs, while the accessibility of the conjugate in a second coated layer promotes effective interaction of the cells with the biofunctional peptide sequences and thus, optimal cell attachment and proliferation of mesenchymal stromal cells as described in next section.

### From Surface Charge to Efficient Cell Culture Coating

It has been found in the examples of the present invention that the change of the zeta potential by application of certain coating layers can be used to estimate the effectiveness of the surface coating and to predict the adherence or binding of cells to the coated article.

Charged surfaces enable a strong interaction with cell surfaces. Therefore, the surface charge of cell culture scaffolds is conventionally enhanced by chemical modification to promote binding of the cells to these articles. It has been applied by the industry as an effective strategy to enable the culture of human and animal-derived cells. However, this procedure has the disadvantage that increased surface charges of the cell culture scaffolds promote an unspecific interaction with the cell surfaces rather than a specific cell binding.

The LbL coating procedure of the present invention is based on interaction of charged polymers with the charged scaffold surfaces, which leads to a reduction of the absolute value of the charge. It has been surprisingly found that, although the surface charge of the coated article is lowered by the LbL coating procedure, the cell binding can be even more increased, and selectivity of the cell binding is enhanced and/or can be controlled. Positive effects on the adherence or binding of cells to the coated article have been found in the present invention, when the surface of the coated article shows a zeta potential in the range between −50 mV and 50 mV, preferably between −30 mV and 30 mV, more preferably between −10 mV and 10 mV, most preferably between −3 mV and 3 mV.

It was found that surface charge is not the dominating criteria and moreover, not the most beneficial to culture high quality stem cells. The stem cells are provided with essential mechanical cues of being bound to a rigid interface but bioactive signals reassembling their niche of origin are missing. Furthermore, detachment and harvest of nonspecifically bound cells require harsh, potentially quality-impacting conditions with conventional coatings. This disadvantage can be overcome with the LbL coating of the present invention.

The presence, concentration, and homogeneous distribution of biofunctional ligands on the scaffold support efficient cell attachment and growth in a biologically relevant manner by stimulation of adhesion receptors. In the given invention, the specific ligand-receptor binding is promoted by the bioactive peptides comprised in the coating, which are responsible for cell binding to the coated article. This is advantageous because it supports cell expansion while maintaining expression of the expected cell morphology, and stem cell identity (see also Examples of the invention, II Results, Section E, as well as FIGS. 9A-9C and FIG. 10). It further enables an enzymatic detachment of the cells from the surface of the coated article proving the low impact on the cells as shown by multi-passage experiments.

In contrast, in the case of a resulting surface charge of the coated article outside the range described above, i.e. in a strongly negative or positive range, the formation of protein-protein-interactions (receptor binding) between surface proteins of the cells and the bioactive peptides comprised in the coating layers is disturbed and charge-charge-interactions, such as ionic bindings, are dominating the cell binding to the coated article. This is disadvantageous, because ligand specific stimulation for maintenance of stemness is missing and the non-specifically bound cells are difficult to detach from the surface of the coated article and cells would rather be damaged or destroyed upon their removal.

Measurements of the surface charge, such as zeta potential measurements are a powerful tool to demonstrate the effective deposition of these biofunctional materials on the coated scaffold. Significant alternations of the zeta potential (Δ≥20 mV) upon each coating step proof the efficiency of deposition of each layer.

In simple words, the present invention provides a solution for masking the initial charge of a cell culture scaffold in order to diminish unspecific binding and cell damage caused by said charge. Functionality, i.e. means for specific cell binding is added to the coating according to the invention by implementing biofunctional peptides.

It is a special advantage of the invention that any cell culture scaffold material can be coated in consideration of its surface charge such that cells can adhere or bind to the coating and that the coated article can be used for cell culturing. Accordingly, a great variety of cell culture scaffold materials can be used within the invention.

Suitably, the cell culture scaffold material is selected from synthetic polymers such as polystyrene, polypropylene, polyester and poly-ethyl(methacrylate) (PEMA), and natural polymers such as polysaccharides like dextran, starch, agarose, and alginate. Any of these materials can be positively charged, negatively charged and neutral depending on their production conditions. Moreover, they can be used in 2D-shape or 3D-shape and as non-porous or porous material or as hollow fiber material. Preferred, according to the invention, are 3D-cell culture scaffold materials, in particular microcarriers. A special advantage of the invention is that 3D-materials can be coated because the coating is gravity-independent and is instead driven by diffusion, concentration gradients, adsorption, und charge interaction, in particular electrostatic interaction.

The coated article of the invention is suitably produced by the so-called LbL coating, which is described herein below. Depending on the charge of the surface of the cell culture scaffold material, the order of the layers coated onto the scaffold material is different.

In a preferred embodiment the invention provides a coated article according to any of the preceding claims, wherein said cell culture scaffold material comprises a negatively charged surface or positively charged surface, which comprises a multilayer coating comprising or consisting of the structure (YZ)ₙ or (YZY)ₙ for the negatively charged scaffold surface; or (ZY)ₙ or (ZYZ)ₙ for a positively charged surface, wherein Y represents a layer comprising or consisting of a positively charged conjugate; Z represents a layer comprising or consisting of at last one negatively charged polysaccharide (NCP); and n is an integer in the range of 1 and 100.

Preferably, n is an integer in the range of 1 to 90, 1 to 80, 1 to 70, 1 to 60, more preferably 1 to 50, or 1 to 40, 1 to 30, or 1 to 20, most preferably 1 to 10, or 1 to 5, especially preferred 1 to 3.

“Structure” means order of the layers relative to the surface of the cell culture scaffold material. For example, in the structure (YZ)ₙ, layer Y is coated onto the surface of the cell culture scaffold material as first layer, and layer Z is subsequently coated onto layer Y. In the structure (ZY)ₙ, layer Z is coated onto the surface of the cell culture scaffold material as first layer, and layer Y is subsequently coated onto layer Z.

The LbL coating of the present invention is especially suitable for the coating of 3D-scaffolds. Most preferred according to invention is therefore a coated article, wherein said cell culture scaffold material has a three-dimensional structure, such as a microcarrier. It may be porous or not.

A further advantage of the LbL coating of the present invention is that the thickness of the layers is very low compared to other coating approaches known in the prior art. Preferably, the typical thickness of one layer is 10 nm or lower, preferably <10 nm, more preferably 9, 8, 7, 6, 5, 4, 3, 2 or 1 nm. This leads to a material saving of 100 to 1,000-fold compared to conventional coating approaches and gives the LbL coating of the present invention an economic advantage.

Layer Y has two functions: It is positively charged to interact with a negatively charged surface of the cell culture scaffold material or with the negatively charged layer Z via charge interactions or adsorption. In order to enable the adhering or binding of cells to the coated article of the invention, layer Y further comprises means for cell binding or interaction with cells.

In a more preferred embodiment, layer Y comprises or consist of a polymer wherein a peptide sequence is coupled to it to create a conjugate, and wherein said peptide sequence consists of a linker peptide (KA)ₙ and a biofunctional peptide. In said (KA)ₙ, K is lysine, A is alanine and n is an integer selected from 3, 4, 5, 6, 7, 8, 9, 10, 11, and 12. In preferred embodiments, n is 5, 7 or 9. So, preferably, the linker peptide is selected from (KA)₅ (SEQ ID NO. 1), (KA)₇ (SEQ ID NO. 2) and (KA)₉ (SEQ ID NO. 3). Most preferably, the linker peptide is (KA)₇ (SEQ ID NO. 2). The linker peptide is suitably positively charged and adds the positive charge to the entire conjugate of layer Y.

In the peptides of the present invention, each amino acid residue is represented by a one-letter or a three-letter designation, corresponding to the trivial name of the amino acid, in accordance with the following conventional list (Table 1):

TABLE 1
Designation of amino acid residues

| Amino Acid | One-Letter Symbol | Three-Letter Symbol |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Said biofunctional peptide is capable of interaction with cells and enables their binding to the coated article of the invention. The biofunctional peptide is preferably selected from the peptides of SEQ ID NOs: 4 to 344. These sequences comprise oligopeptide sequences which mimic the ECM and signal peptides. More preferably, the biofunctional peptide is one of those shown in Table 2. These sequences represent binding sites from proteins of the extracellular matrix, which are most relevant for primary and stem cells, e.g., laminin, fibronectin, vitronectin, bone sial protein, collagen, cadherin and tenascin.

TABLE 2
List of most preferred peptide sequences.

| Biofunctional Peptide | SEQ ID NO |
|---|---|
| KA5 | 1 |
| KA7 | 2 |
| KA9 | 3 |
| RGDSP | 4 |
| FRLVFRY | 5 |
| NGEPRGDTYRAY | 6 |
| NPWHSIYITRFG | 7 |
| PHSRN | 8 |
| PHSRNGGRGDSP | 9 |
| RGD | 10 |
| CSRNLSEIKLLISRARK | 11 |
| DVDVPDGRGDSLAYG | 12 |
| EIKLLIS | 13 |
| GEFYFDLRLKGDKY | 14 |
| GFPGER | 15 |
| LEPRREVCELNPD | 16 |
| LFSHAVSSN | 17 |
| LGGLPSHYRARNI | 18 |
| NRWHSIYITRFG | 19 |
| PQVTRGDVFTMP | 20 |
| RKIPKASSVPTELSAISMLYL | 21 |
| SIDRVEPYSSTAQ | 22 |
| TFDLLRNSYGVRK | 23 |
| TWSKVGGHLRPGIVQSG | 24 |
| TWYKIAFQRNRK | 25 |
| VAEIDGIELT | 26 |
| VFDNFVLK | 27 |
| QPPRARI | 28 |
| YRSRKYSSWYVALKRK | 29 |
| ESPLKRQ | 30 |

Most preferred according to the invention are the biofunctional peptides selected from the group consisting of SEQ ID NOs: 4, 5, 6, 7, 8 and 9. These peptide sequences are most important for the culture of stem cells like mesenchymal stem cells and induced pluripotent stem cells and cell types derived thereof and are of special interest for the application of this invention.

In a further preferred embodiment of the invention, the polymer used in layer Y is polyethylene glycol (PEG). PEG is an oligomer or polymer composed of ethylene oxide monomers. Because different applications require different polymer chain lengths, PEGs are prepared by polymerization of ethylene oxide and are commercially available over a wide range of molecular weights from 300 g/mol to 10,000,000 g/mol. While PEGs with different molecular weights find use in different applications, and have different physical properties (e.g. viscosity) due to chain length effects, their chemical properties are nearly identical.

PEGs are also available with different geometries:
Linear PEGs, where the ethylene oxide monomers are bound to each other in an unbranched polymer chain;
Branched PEGs, which have three to ten PEG chains emanating from a central core group;
Star PEGs, which have 10 to 100 PEG chains emanating from a central core group; and
Comb PEGs, which have multiple PEG chains normally grafted onto a polymer backbone.

The numbers that are often included in the names of PEGs indicate their average molecular weights (i.e. a PEG with n=9 would have an average molecular weight of approximately 400 Da and would be labeled PEG 400). Most PEGs include molecules with a distribution of molecular weights (i.e. they are polydisperse). The size distribution can be characterized statistically by its weight average molecular weight (M_{w}) and its number average molecular weight (Mₙ), the ratio of which is called the polydispersity index (M_{w}/Mₙ). M_{w} and Mₙ can be measured by mass spectrometry.

PEG is soluble in water, methanol, ethanol, acetonitrile, benzene, and dichloromethane, and is insoluble in diethyl ether and hexane.

In a preferred embodiment, layer Y of the coated article of the invention comprises a starPEG. Suitably, said starPEG has a molecular weight in the range of 4 kDa to 40 kDa, preferably in the range of 4 kDa to 30 kDa, more preferably in the range of 4 kDa to 20 kDa, most preferably in the range of 4 kDa to 10 kDa. Further most preferably, said starPEG is a 4-arm starPEG. The use of 4-arm starPEG in the hydrogels according to the invention has been found to be especially suitable when a peptide sequence is coupled to each of the four arms, and when said peptide sequence consists of a linker peptide (KA)ₙ and a biofunctional peptide as described above.

More preferably, layer Y of the coated article of the invention comprises a linear PEG. Linear PEGs have the advantage of being cheaper and possess a narrower molecular weight distribution. The use of a linear PEG in layer Y of the coated article according to the invention has been found to be especially suitable, when a peptide sequence is coupled to each of both ends, and when said peptide sequence consists of a linker peptide (KA)ₙ and a biofunctional peptide as described above. By using linear PEG instead of starPEG, the charge density of the positive charges in the conjugate of layer Y is higher, i.e. the portion of the uncharged structure-giving polymer is lower, which saves material and costs.

Most preferably, the linear PEG comprised in layer Y of the coated article according to the invention has a molecular weight in the range of 1 kDa to 100 kDa, preferably in the range 2 kDa to 80 kDa, 3 kDa to 60 kDa, 4 kDa to 40 kDa, most preferably in the range of 5 kDa to 20 kDa. Even most preferably, the linear PEG comprised in the conjugate according to the invention has a molecular weight selected from 5 kDa, 10 kDa, 15 kDa and 20 kDa.

In a further preferred embodiment, the PEG, which is used to prepare layer Y of the coated article of the invention, is functionalized. It is meant by “functionalized” to modify a molecule in a manner that results in the attachment of a functional group or moiety. For example, a molecule may be functionalized by the introduction of a functional group or structural feature, which makes the molecule a strong nucleophile or a conjugated unsaturation. Preferably a molecule, for example PEG, is functionalized to become a thiol, amine, acrylate, azide, alkyne, or quinone. More preferably, for use in the preparation of the conjugate of the invention, the PEG is maleimide-functionalized, carboxylic acid-functionalized, amino-functionalized, azide-functionalized, or alkyne-functionalized.

Said negatively charged polysaccharide (NCP) of layer Z interacts with the positively charged surface of the cell culture scaffold material or with the positively charged layer Y via charge interaction or adsorption and is preferably selected from poly(sodium-4-styrenesulfonate) (PSS) or a sulfated or phosphorylated oligosaccharide, preferably selected from a group consisting of polysaccharides which comprises heparin, heparan sulfate, chondroitin sulfate, dermatan sulfate and keratin sulfate, dextran sulfate, α-cyclodextrin sulfate, β-cyclodextrin sulfate, γ-cyclodextrin sulfate, α-cyclodextrin phosphate, β-cyclodextrin phosphate, and γ-cyclodextrin phosphate.

In an especially preferred embodiment the NCP is heparin, which originates from the mucosa of pig intestine or bovine lung tissue. Heparin is preferably of pharmaceutical quality. In a most preferred embodiment, the NCP is dextran sulfate. Said dextran sulfate has preferably a molecular weight in the range of 4 kDa to 600 kDa. Preferred is the use of dextran sulfate of pharmaceutical quality. If layer Z contains cyclodextrin sulfate, then it is preferably α-cyclodextrin sulfate, β-cyclodextrin sulfate, γ-cyclodextrin sulfate of pharmaceutical quality, wherein the sulphation degree is of three sulfates per molecule up to a complete sulphation degree. If layer Z contains α-cyclodextrin phosphate, β-cyclodextrin phosphate, γ-cyclodextrin phosphate then it is of pharmaceutical quality, wherein the degree of phosphorylation of three phosphate groups per molecule can be up to the complete phosphorylation.

In a further preferred embodiment, the multilayer coating used in the LbL approach of the invention does not comprise chitin together with carrageenan, such as a κ-, l- or λ-carrageenan (kappa-, iota- or lambda-carrageenan). The combination of these polysaccharides is used by Oliveira et al.²⁵ to produce nanocoatings containing sulfated polysaccharides prepared by LbL assembly as models to study cell-material interactions.

In a further embodiment, the coated article of the invention may comprise a cell culture scaffold material with a neutral (i.e. uncharged) surface. In this case, the coating of the coated article may further comprise a primer layer comprising or consisting of a charge-inducing component, which adds a positive charge to the surface of the uncharged cell culture scaffold material for further coating with the layers Y and Z.

Accordingly, in a further preferred embodiment, the invention provides a coated article, wherein said cell culture scaffold material comprises a neutral (uncharged) surface, which comprises a coating comprising or consisting of:
i. a layer comprising or consisting of a charge-inducing component as primer layer; and
ii. a multilayer coating comprising or consisting of the structure (ZY)ₙ or (ZYZ)ₙ as defined herein above.

The charge-inducing component is preferably selected from poly-L-lysine, poly-D-lysine, poly-ornithine or other positively charged polypeptides.

It is a special advantage of the invention that the coated article as described herein is obtainable by LbL coating, which is driven by charge-based interactions and/or adsorption between the surface of the cell culture scaffold material and the coating layers.

In a further embodiment, the coated article of the invention may comprise cells. The cells are typically eukaryotic cells, preferably from vertebrates and anthropoids, preferably mammalian cells, most preferably human cells, or human cell lines. In a more preferred embodiment, the cells are pluripotent stem cells especially embryonic and induced pluripotent stem cell (iPSC) and all cell types derived thereof, and all primary adult stem and progenitor cells especially satellite cells, bone marrow derived mesenchymal stem cells (bmMSC), umbilical cord derived mesenchymal stem cells (ucMSC), and adipose tissue derived mesenchymal stem cells (atMSC), and any cell type thereof, myoblasts and adipose progenitor cells, as well as primary somatic cells like fibroblasts, endothelial and epithelial cells. Most preferably, the cells are primary cells or stem cells.

Typically, the cells interact with the biofunctional peptide of the conjugate of layer Y and adhere thereby to the coated article of the invention.

The biofunctional peptide can include an amino sequence derived from ECM proteins such as collagen, fibronectin, vitronectin, laminin, bone sialo protein, or the like, or portions thereof. In embodiments, the biofunctional peptide includes an amino acid sequence of Arg-Gly-Asp (RGD) or a derivative cell adhesive sequence, recognized by cell adhesion receptors like the integrin family or other molecules leading to cell adhesion.

### Methods

In current cell culture practice, it is known that specific cell types require the presence of a specific adhesion peptide or combination of peptides on the culture surface to enable adhesion and sustainable culture. For example, iPSC prefer laminin or vitronectin derived peptide sequences such as the peptide NGEPRGDTYRAY of SEQ ID NO. 6. The invention further provides a method for preparing the coated article of the invention. It should be recognized that the advantages and advantageous embodiments described above for the coated article according to the invention equally apply to the method for preparing of said coated article such that it shall be referred to the above.

In particular, a method is provided for preparing a coated article comprising a cell culture scaffold material with a non-covalent coating according to any one of the preceding claims, comprising the steps of
i. selecting a cell culture scaffold material;
ii. estimating the charge of the surface of the cell culture scaffold material, and
iii. depending on the charge of the surface of the cell culture scaffold, coating said cell culture scaffold with a multilayer coating comprising or consisting of the structure (YZ)ₙ or (YZY)ₙ for a negatively charged surface; or (ZY)ₙ or (ZYZ)ₙ for a positively charged surface, wherein
Y represents a layer comprising or consisting of a positively charged conjugate;
Z represents a layer comprising or consisting of at last one negatively charged polysaccharide (NCP); and
n is an integer in the range of 1 and 100.

When said cell culture scaffold material comprises a neutral (uncharged) surface, it is coated with
a layer comprising or consisting of a charge-inducing component as primer layer; and
a multilayer coating comprising or consisting of the structure (ZY)ₙ, or (ZYZ)ₙ as defined herein.

The method according to the invention is advantageous, because the LbL deposition on the cell culture scaffold material is driven by adsorption und/or charge interaction between the surface of the cell culture scaffold material and the coating layers. In particular, the method of the invention is gravity-independent and therefore suitable for coating of 3D-shaped as well as porous structures.

Estimation of the charge of a scaffold can be performed e.g. by measuring the zeta potential or by labeling the scaffold with charged biomaterials and examining the scaffold with fluorescence microscopy, as described hereinabove.

The coating of 2D cell culture plastic ware and 3D scaffolds can be performed using the LbL approach according to the invention. For example, diluted aqueous solutions (1 to 1,000 μM) of NCPs and positively charged conjugates in a suitable buffer, such as PBS buffer are prepared as coating solutions. Coating of plastic ware in 2D is performed by filling the well/flask with a first coating solution. In case of 3D scaffolds, samples are immersed in the first coating solution and gentle swirling was applied. A duration of 10 to 60 min of coating duration is suitably applied. Thereafter, the materials are washed twice with water, preferably deionized water. Subsequently, the second coating solution is added, and the procedure is repeated likewise for every following coating step. Upon completion, coated articles were left for drying.

The present invention is based on a LbL coating, that presents a biologically functional improvement of existing solutions for use in industrial-scale cell manufacturing for regenerative medicine and cellular agriculture. The introduction of novel combinations of peptide sequences allows for culturing of sensitive anchorage-dependent cell types like stem cells and tissue cells derived thereof. The simple coating protocol developed can be applied off- and online and as off-the-shelf pre-coated products optimally suited for industrial processes. The novel coating protocol allows its application onto various types of material, in particular natural scaffolds, without further chemical modifications like crosslinking.

In a further step, cells may be adhered to the coated article by incubating the coated article in a culture medium comprising cells, preferably primary cells and/or stem cells, most preferably of human origin.

The invention further relates to the use of the coated article according to any one of the preceding claims in a method for culturing cells, preferably primary cells and/or stem cells, most preferably of human origin. In general, the coated article would also be suitable for the culturing of any type of eukaryotic cells.

In a further embodiment, the invention provides the coated article with cultured cells adhered to it for use in regenerative medicine and in cellular agriculture.

Regenerative and aesthetic medicine defines a medical treatment of any type of regeneration, supplementation, or replacement of cells, organs or tissues with cells, engineered cell-tissue or cultured organs or similar living therapeutics.

Cellular agriculture is defined as production of agriculture products from cell cultures derived from wild or domesticated animals.

In a further embodiment, the invention relates to the use of the coated article with cultured cells adhered to it for the preparation of a cell and gene therapy products for the treatment of diseases or disorders in the field of regenerative medicine, like cardiac diseases e.g. hear failure, ophthalmic diseases e.g. retinal degenerations, macular degeneration/dystrophy, optic atrophy and retinopathy, ocular diseases; neurologic disorders e.g. Parkinson's disease, spinal cord injury, diabetes; blood diseases e.g. thalassemia, Sickle-cell disease; cancer.

In a further embodiment, the invention relates to the use of the coated article with cultured cells, preferably stem cells adhered to it in aesthetic medicine, such as aesthetic dermatology and plastic surgery procedures, e.g. for application in dermatology (e.g. burns, scars, wrinkles, abnormalities) or plastic surgery to support wound healing process after filler injections, breast augmentation, or for supporting fat-grafting.

In a further embodiment, the invention relates to a method of treatment of diseases or disorders in the field of regenerative medicine comprising the administration of a therapeutically effective amount of the coated article with cultured cells adhered to it to a subject in need thereof.

In a further embodiment, the invention relates to cells derived from wild or domesticated animals of the myogenic lineage, e.g. myogenic progenitor, adipocytes, myoblast, satellite cells, skeletal muscle, cardiac muscle, smooth muscle as well as white adipocytes. In addition, the invention relates to MSCs and iPSCs and their derivatives of animal species origin, such as bovine, porcine, poultry, salmon, and tuna.

The invention is further illustrated by certain drawings and examples.

### BRIEF DESCRIPTION OF THE DRAWINGS

FIGS. 1A and 1B show schematic sketches of the two main components used in the LbL approach. The positively charged conjugate consists of a star-PEG backbone conjugated to a linker peptide and a biofunctional peptide. The negatively charged polysaccharide (NCP) is a highly sulfated sugar molecule.

FIGS. 1C-1E show schematic coating strategies for three differently charged scaffolds (positive, negative, neutral). Depending on the surface charge of the scaffold the coating order is adapted. Corresponding results from zeta potential measurements on a bead-based model system confirm as a proof of principle the effectiveness of each strategy.

FIG. 2 shows the change of zeta potentials of positively charged microcarriers in dependence of the individual coating components with different concentrations. The LbL coating was first coated with an NCP followed by a conjugate coating.

FIGS. 3A-3C show the fluorescence quantification of fluorescent NCP (FITC-NCP) or fluorescent conjugate (Cy5-conj.) deposited on positively charged microcarrier in relation to coating concentration. FIG. 3A) Fluorescence intensity of a positively charged microcarrier coated with different concentrations of FITC-NCP. FIG. 3B) Fluorescence intensity of a positively charged microcarrier coated with NCP and subsequently coated with different concentrations of Cy5-conjugate. FIG. 3C) Fluorescence microscope image series of microcarriers coated with FITC-NCP (top) and Cy5-conjugate (bottom). The concentration of the coating solution increases from left to right. Scale bar: 500 μm.

FIG. 4 shows the quantification of deposited conjugate on microcarriers by photometric BCA assay in dependency of the coating solution concentration (0-50 PM). The overall conjugate concentration deposited on the beads is determined by the UV-Vis absorption of a resulting copper complex at 562 nm.

FIG. 5 shows fluorescence microscope images of 2D cell culture plastic ware partially coated with fluorescently labelled TRITC-NCP (5% dye content) or Cy5-conjugate (50% dye content). The partial coating was achieved by dropcasting small volumes of coating solution on the plastic ware which creates a sharp contrast between coated and uncoated surface. The dashed lines highlight the edge of the coated area. The coating has been performed consecutively using LbL with NCP as first layer followed by conjugate. Scale bar: 500 μm.

FIG. 6 shows that multiple scaffold materials can be coated with the LbL coating technology of the invention, exemplified by natural polymers alginate, agarose, dextran, starch, and cellulose and the synthetic polymers polystyrene and polypropylene/polyester. The coating efficiency was tested by fluorescence microscopy of the scaffolds using fluorescently labelled FITC-NCP and Cy5-conjugate. Scale bar: 500 μm. A quantitative evaluation of the mean fluorescence intensity of the labeled components (grey columns) compared with the background signal (black columns) is given in the graphs below.

FIGS. 7A and 7B show a comparison of the applicability of different primers. Poly-D-lysine (PDL), poly-L-lysine (PLL) and poly-L-ornithine (PLO) have been tested as an initial primer to enable the coating on neutrally charged scaffolds. FIG. 7A) The three different primers were applied on polystyrene microcarriers and fluorescently labelled FITC-NCP and Cy5-conjugate added consecutively. Scale bar: 500 μm. FIG. 7B) A numeric evaluation of the microscopy data comparing the mean fluorescence intensity of the NPC and conjugate of coated scaffolds in dependency of the applied primer type.

FIGS. 8A-8C show that different parameters of LbL coating are tunable and contribute to optimizing cell response. Negatively charged polysaccharides (NPC) and positively charged conjugates (Conj) were deposited in different numbers of coating cycles, orders, and concentrations on 2D cell culture plates. FIG. 8A) Response of mesenchymal stromal cells (MSC) to different number of layers deposited in an LbL approach in comparison to a coacervation based control (n=3, tr=3). FIG. 8B) The impact of different orders of component deposition is shown for cell growth of MSC in comparison to a coacervation based control (n=3, tr=3). FIG. 8C) Cell growth of induced pluripotent stem cells (iPSCs) on LbL coatings with varying coating solution concentrations (n=2, tr=5).

FIGS. 9A-9C show that stem cells require specific coating formulations for optimal cell growth. FIG. 9A) Overall cell growth of iPSCs on plates coated with either an optimized formulation of NPC and conjugate (iPSC Matrix) using our LbL method or the previously reported coating strategy and formulation (RGDSP) (n=3, p<0.0001). FIG. 9B) Visualization with crystal violet of cell growth on an entirely coated well using iPSC Matrix or RGDSP formulation. FIG. 9C) Representative phase-contrast images of cell colonies grown on LbL coatings with either iPSC Matrix or RGDSP formulation. Scale bar: 100 μm.

FIG. 10 shows stem cell growth on glass that was either covalently modified with previously reported formulation RGDSP-Heparin (Covalent RGDSP) or coated with the LbL technique with PDL as a primer followed by an optimized formulation of layers of NCP and conjugate (iPSC MATRIX). Shown are iPSCs in phase contrast or stained with stemness marker OCT4 and DAPI (nuclei). Scale bar: 100 μm.

FIG. 11 shows the in vitro stabilization of bFGF by NCP coating component. mTESR1™ media relevant for iPSC growth was incubated at 37° C., 5% CO₂, for different periods on the coating of the invention and another animal-component free coating which does not contain NCP as a component. The concentration of bFGF present in the media was determined with an ELISA assay. The data was fitted using one phase decay model: Y=(Y0−Plateau)*exp(−K*X)+Plateau, coating r²=0.905 and non-NCP coating r²=0.9914.

FIGS. 12A-12C show that LbL coating allows for 3D cultivation of mesenchymal stromal cells. FIG. 12A) Glucose consumption over time on beads either coated with NCP-Conj or Conj-NCP-Conj. Arrows indicate media change. FIG. 12B) Fold expansion of cell number seeded onto microcarriers with different coating layer numbers after 11 days of cultivation. FIG. 12C) Images of cells cultivated on beads in Erlenmeyer flasks. The formation of larger cell-microcarrier aggregates on Conj-NCP-Conj is highlighted in the magnified image and illustrated in the fluorescence microscope images. Scale bar: 100 μm

### DETAILED DESCRIPTION

### I. MATERIAL AND METHODS

### LbL Coating Procedure

Coating of 2D cell culture plasticware and 3D scaffolds was performed using the LbL approach. Dilute aqueous solutions (0.5-50 μM) of NCPs and conjugates in PBS buffer were used as coating solutions. Coating of plasticware in 2D was performed by filling the well/flask with the first coating solution. In the case of 3D scaffolds, samples have been immersed in the first coating solution and gentle swirling was applied. In both 2D and 3D, 30-60 min of coating duration was applied. Afterwards, samples were washed twice with deionized water. Subsequently, the second coating solution was added, and the procedure was repeated likewise for every following coating step. Upon completion samples were left for drying.

### Zeta Potential Measurements

Zeta potential measurements were carried out on a Zetasizer Nano ZS (Malvern Panalytical). Positively and negatively charged polystyrene beads were purchased from Polysciences Inc. Neutral beads were supplied by Surflay Nanotec GmbH. For zeta potential measurements, beads were suspended in dilute NaCl solutions (c=1 mM) and suspensions were filled into Folded Capillary Zeta Cells (Malvern Panalytical). The electrophoretic mobility of the particles is measured using a laser Doppler velocimeter. Zeta potential values were approximated using the Smoluchowski model included in the Zetasizer Nano software (Malvern Panalytical). For the coating titration experiments, positively and negatively charged polystyrene beads were coated with solutions of NCPs and conjugates in dilute aqueous NaCl solutions (c=1 mM) instead of PBS buffer.

### Fluorescent Imaging of Coated 2D and 3D Scaffolds

2D and 3D scaffolds used for fluorescent imaging were coated using the LbL coating procedure. All coatings contained 5% of a Cy5-fluorescently labeled conjugate and the NCP contained 50% of either a TRITC-fluorescently labeled NCP (for 2D assessment) or a FITC-fluorescently labeled NCP (for 3D assessment). Fluorescence imaging was carried out using a Lionheart FX automated microscope (Agilent) and settings were kept equally in each experiment. Fluorescence intensity was quantified using the Gen5 software (BioTek) Version 3.10.06 and statistical processing performed using the GraphPad Prism software version 9.3.1.

### Bicinchoninic Acid (BCA) Peptide Assay

Conjugates deposited on coated beads have been quantified using the colorimetric BCA assay. The BCA working solution (ThermoFisher Scientific) was added to the coated beads and the mixture was incubated for 2 h at 37° C. while constantly inverted. Subsequently, the beads were cooled using an ice bath and the beads were separated from the analyte solution using a strainer (40 μm mesh). The peptide concentration was determined photometrically using absorption at 562 nm using a UV-3100PC spectrophotometer (VWR).

Covalently Coating of Glass Slides with Heparin

Glass slides have been covalently functionalized according to the prior art¹⁸ to compare their performance with LbL-coated glass slides according to the present invention. In short, glass slides have been cleaned by ultrasonication and rinsing with deionized H₂O, acetone, and piranha solution (H₂O₂+H₂SO₄ 1:3). The blow-dried slides were then aminosilanized using (3-Aminopropyl)triethoxysilane (APTES, VWR). Heparin was covalently bound to amino groups on the glass slide by activation with 1-Ethyl-3-(3-dimethylaminopropyl)carbodiimide (EDC, VWR) and N-Hydroxysulphosuccinimide (sulfo-NHS, VWR). A layer of conjugate RGDSP (see Table 2, ID #4) was attached by LbL coating. For comparison, glass slides were noncovalently coated with an initial PDL layer, followed by an NCP layer and a conjugate layer (see Table 2, ID #6) following the LbL approach of the invention. All glass slides were placed in a 24-well plate for cell culture testing.

### Cell Culture Testing—Growth Experiments for Coating Optimization

Human iPSCs were cultured in mTeSR™1 (STEMCELL Technologies) on different surfaces at 37° C. and 5% CO2 in a humidified incubator. For each experiment cells were dissociated using ReLeSR™ (STEMCELL Technologies) for 3-5 min following the manufacturer's protocol. The resulting clumps were seeded onto the test plates and grown for 4 days. Human bone marrow derived MSC (hBM-MSC) were cultured in StemPro (StemPro™ MSC SFM, Gibco, #A1033201) media. Cells were seeded at a density of 5.000 cells/cm² into 24-well plates and cultured until 90% confluency at 37° C. and 5% CO₂ in a humidified incubator. After fixation with 4% PFA, cells were stained with 0.5% Crystal violet for 10-20 min, then washed and dried at room temperature. The crystal violet stain was subsequently released from the cells in order to quantify cell numbers using absorbance spectroscopy.

bFGF Concentration Determination

The concentration of bFGF in mTESR™1 after incubation for different time points at 37° C. was determined using the ELISA kit for human bFGF (Sigma Aldrich, catalog no. RAB0182-1KT). Briefly, all components of the mTESR™ 1 kit were combined and incubated in 6-well plates that were previously coated with the coating and another animal-component free coating that does not contain NCP as a component. Plates were placed into a cell culture incubator for 24, 48, 72, and 96 hours at 37° C., 5% CO₂. The ELISA was performed according to the manufacturer's instructions using the provided protein standard. Each measure was run in duplicate and the experiment was performed in triplicate.

### 3D Cultivation of Mesenchymal Stromal Cells

Human bone marrow derived MSCs (hBM-MSC) were cultured in StemPro (StemPro™ MSC SFM, Gibco, #A1033201) media. Cells were seeded at a density of 4,000-5,000 cells/cm² into Erlenmeyer flasks containing 15 g/L coated or non-coated microcarriers. After a static attachment period overnight at 37° C. and 5% C02 in a humidified incubator, flasks were shaken at 90-110 rpm over the course of 11 days. Glucose consumption was measured using the BIOSEN C-line device (EKF diagnostics). Cells were detached by adding TrypLE™ Express Enzyme (Gibco™) and Collagenase for 20 min and counted using the Eve™ NanoEntek cell counter.

### II. RESULTS

### A) Deposition of the LbL Coating is Driven by Charged-Based Interactions

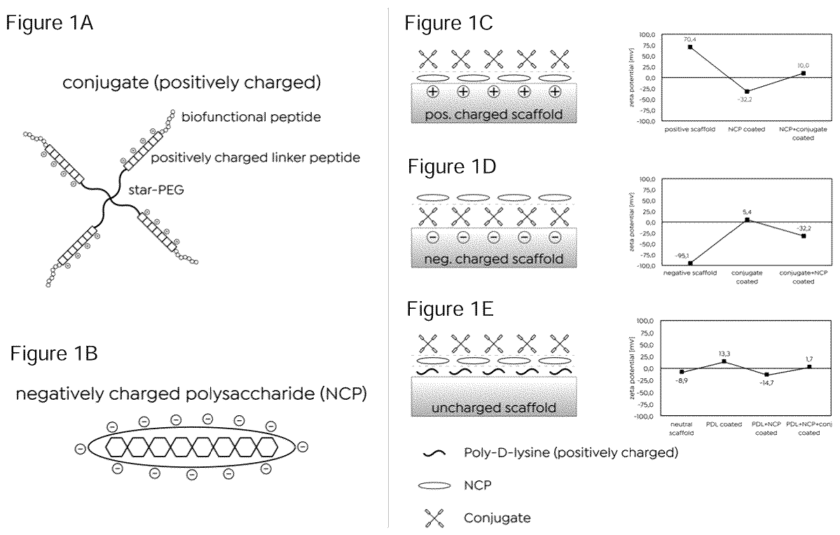

The coating of cell culture ware and scaffolds is carried out using the LbL approach. The driving force of this deposition process is electrostatic interaction¹⁹. Thus, charged molecules are particularly suitable for LbL processes. Here, a method is provided employing two classes of oppositely charged biofunctional components to build effective coatings—a positively charged peptide-polymer-conjugate (FIG. 1A, in the following referred to as “conjugate”) and a negatively charged polysaccharide (NCP) (FIG. 1B).

The conjugate consists of a 4-arm star-shaped PEG molecule, which acts as the structural backbone of the coating. To each of the four arms, a peptide sequence is coupled. The sequence consists of a linker peptide and a biofunctional peptide. The linker peptide consists of repetitive units of lysin (K) and alanine (A), generating a positive molecular charge. The linker sequence has been designed to connect effectively to NCP²⁰. The biofunctional peptide provides cells with enhanced and selective attachment to the coating (see sequences list). It has been reported that by selection of linker and biofunctional peptide sequence, optimal cell attachment and growth is supported in a cell type specific manner^{13,17}.

The NCP is a highly sulfated polysaccharide molecule, carrying a high density of negative charged moieties. Commonly, heparin has been used, a glycosaminoglycan (GAG) obtained as a naturally occurring product from different animal sources. Alternatively, synthetic dextran sulfate can be employed as animal-component free alternative, which qualifies the so-formed coating for applications in cell culture, regenerative medicine, and cellular agriculture.

Exploiting the interaction of the two components classes, coating strategies were developed based on the LbL approach to address various types of scaffolds with different surface charges. FIGS. 1C-1E describes schematically applicable strategies for negative, positive, and neutral charged scaffold surfaces. The effectiveness of each of these strategies was proven on a model scaffold by zeta potential measurements. As model scaffolds, aqueous suspensions of polystyrene microbeads (d=2-3 μm) with positive (FIG. 1C), negative (FIG. 1D) or neutral surface charge (FIG. 1E) were used. The effectiveness of the deposition was monitored by zeta potential measurements. The corresponding plots indicate that the surface charge alternates strongly with each coating step demonstrating that the LbL deposition of both NCP and conjugate is effective. For a positively charged microbead, the first coating layer is an NCP followed by a second layer of conjugate. A first layer of conjugate is applied on a negatively charged microbead, followed by a second layer of NCP. Consequently, the zeta potentials show a strong alternation with each coating step underlining the effectiveness of the proposed charged-based strategy. In the case of a neutral surface, a first layer of a charge-inducing component, referred to as primer, such as poly-D-lysine (PDL), can create a highly positive charge on the substrate. Poly-D-lysine is commonly used and known to be an effective material to coat tissue culture ware²¹. The PDL layer is followed by a second layer of NCP (layer Z) and a third layer of conjugate as previously proposed for positively charged surfaces (layer Y). The corresponding zeta potential measurement indicates that upon coating with PDL a positive surface charge is induced on the initially uncharged microbead. Consequently, this provides a starting point to enable an effective coating with NCP (layer Z) and conjugate (layer Y).

A positively charged scaffold is the most preferred base material for the LbL coating approach of the invention. It enables an effective deposition of NCPs, while the accessibility of the conjugate in a second coated layer promotes effective interaction of the cells with the biofunctional peptide sequences and thus, optimal cell attachment and proliferation of mesenchymal stromal cells as described in the next section.

To summarize, a charge-based strategy is provided to functionalize differently charged scaffolds with a non-covalent coating consisting of a conjugate and an NCP. This strategy shows broad versatility and applicability.

### Zeta Potential, Fluorescence Microscopy and BCA Assay—Analytical Tools to Monitor and Quantify the LbL Deposition

The coating strategy presented in this invention creates a cell-responsive surface on scaffolds, which enables their use in cell culture. The analysis of cell attachment and growth on the coated surfaces has been used as a quality control of effectiveness and functionality of the given coating. Further analysis methods were qualified that are robust, fast, and simple to monitor and quantify the LbL deposition and for use as routine quality control techniques.

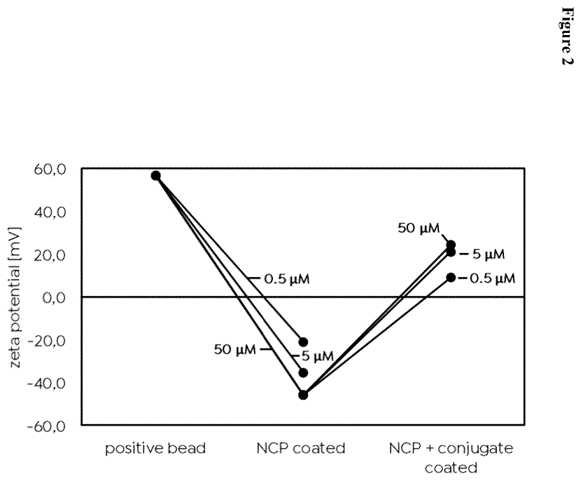

As introduced above, zeta potential (ZP) measurements give insight to the coating process and allow quantification of the deposited material. As a proof of principle, small positively charged polystyrene microspheres (d=3 μm) have been coated with negatively charged NCP followed by a coating with the positively charged conjugate and their ZP was determined (FIG. 2). The molar concentration of both NCP and conjugate in the coating solution was varied between 0.5 and 50 μM. It is found that the initially highly positive surface charge (>50 mV) of the microspheres drops significantly to negative values upon coating with NCP. Moreover, the resulting ZP is influenced by the molar concentration of the applied coating solution, i.e., a higher concentration of NCP results in a more pronounced drop of the ZP to negative values. Accordingly, in a second coating step conjugate was deposited on the NCP-coated (50 μM) microspheres with a variation in the molar concentration between 0.5 and 50 μM. A significant change of up to 70 mV to positive ZP values is observed upon this coating with a more pronounced effect for the higher concentrated conjugate solution (50 βM). In summary, it has been shown that ZP measurements are a powerful tool to monitor the coating process, to give insight in the evolution of the surface charge of the coated microcarrier and to quantify the coating concentration on the beads over two orders of magnitude (0.5 to 50 βM).

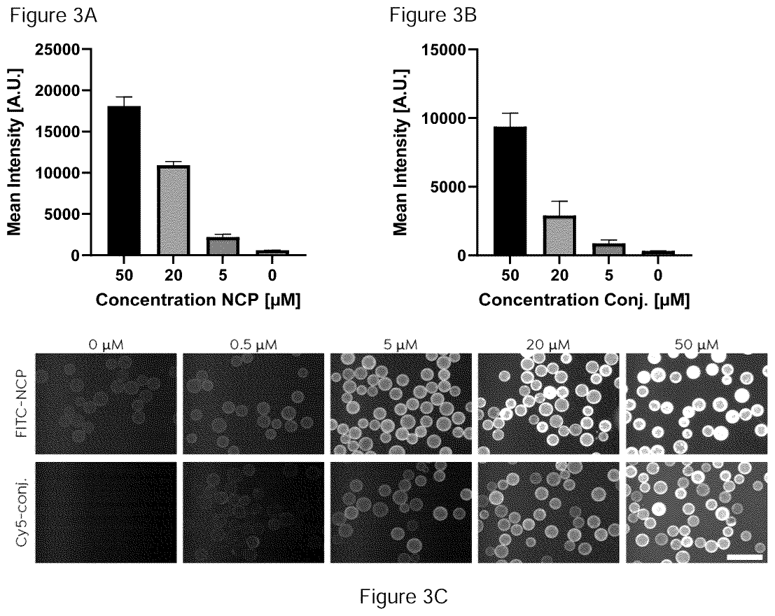

A major drawback of ZP measurement is its limitation to spherical particles with a diameter below 100 μm. Microcarriers, scaffolds or 2D surfaces considered for cell culture do commonly not fulfill these requirements, i.e. cannot be examined with ZP measurements. these limitations fluorescence microscopy has been employed as a versatile tool monitor and quantify the coating process. For this the two main coating constituents NCP and conjugate have been chemically modified with a fluorescent dye label. On NCP fluorescein-isothiocyanate (FITC) or tetramethylrhodamine-isothiocyanate (TRITC) were chosen, on the conjugate the cyanine dye Cy5 was chosen. Small portions of these labelled constituents were added to the regular LbL coating procedure (1^{st} layer NCP, 2^{nd} layer conjugate) on positively charged polystyrene microcarriers (d=170 μm). The coating solutions were varied in concentration and the results of fluorescence microscopy are displayed in FIGS. 3A-3C. FIGS. 3A and 3B show the statistical evaluation of the fluorescence intensity for the differently coated microcarriers for both NCP and conjugate, respectively. In both cases, the mean intensity increases with increasing coating concentration which is in agreement with the previously shown ZP measurement results. Fluorescent microscopy images for the differently coated samples are shown in FIG. 3C. They clearly visualize the increasing fluorescence intensity with increasing coating concentration for both coating steps (top: NCP, bottom: conjugate).

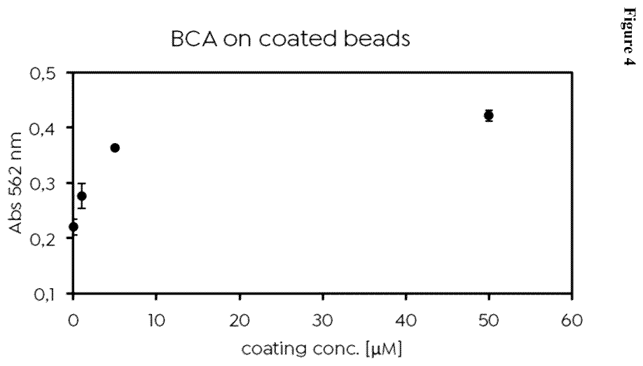

An alternative analysis technique to investigate the coating efficiency and quantify the deposition is the photometric BCA assay. This wet-chemical assay is selective for peptide bonds within proteins and peptides. As a consequence, only the conjugate component of coating strategy of the invention can be analyzed, NCP does not show a specific response to this assay. The BCA analysis does not have specific requirements on the dimension or macroscopic structure of the coated scaffold, however protein-based scaffolds cannot be examined due to their chemical composition. In a model experiment, positively charged polystyrene microcarriers (d=170 μm) have been coated with NCP and in a second step with conjugate solution of varying concentration (0.5 to 50 μM). Performing the BCA assay on these coated microcarriers leads to the results displayed in FIG. 4. With increasing conjugate concentration, a higher absorption at 562 nm is detected, i.e., more conjugate is deposited on the microcarrier surface. These findings are in accordance with the results from ZP measurements and fluorescence microscopy presented previously.

To summarize, there are presented three analytical tools which allow monitoring, quantifying, and visualizing the efficiency of the invented coating process. Moreover, these tools give insight in the surface charge evolution of scaffolds during the coating process and provide information for toxicologic discussions. The presented methods are fast, cheap and robust and can be installed as routine quality control processes.

### Versatility of the Invention—Coating of a Multitude of Natural and Synthetic Natural Microcarriers and Scaffolds

In the previous sections the coating strategy of this invention was presented exemplarily only on spherical polystyrene microcarriers. However, cell culture scaffolds comprise a multitude of different materials, surface properties and morphologies. Thus, fluorescence microscopy was chosen as the analytical tool as it is independently applicable to these multiple varieties.

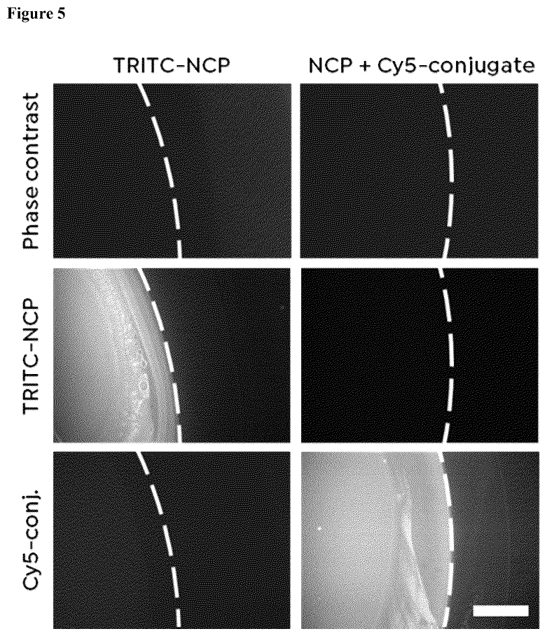

A widely used cell culture scaffold is 2D polystyrene plastic ware such as well plates or T-flasks. In FIG. 5, the effective application of LbL approach of the invention on these scaffolds with NCP and conjugate both labelled with a fluorescent dye is proven by fluorescence microscopy images. Small droplets of coating solution were deposited on the surface and after washing a strong contrast is observed indicating the border between coated and uncoated areas (highlighted with a dashed line).

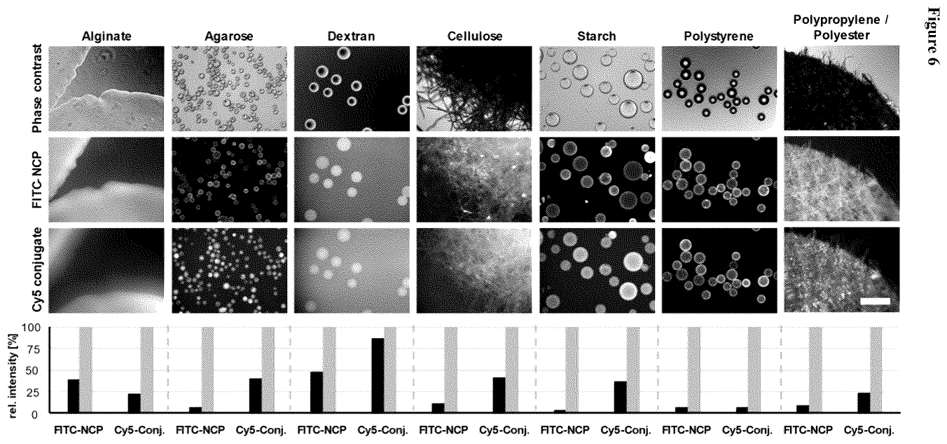

To underline the versatility of the coating procedure presented in this invention, various natural and synthetic materials have been successfully coated. FIG. 6 shows the results of the fluorescence microscopy analysis on cell culture-relevant scaffolds made from the natural polymers alginate, agarose, dextran, cellulose, and starch as well as made from the synthetic polymers polystyrene and polypropylene/polyester copolymer. The morphology of these scaffolds comprises microcarriers of different sizes, fibers and 3D networks. However, the fluorescent assay proves the high efficiency of the inventive coating approach on all materials. This is underlined by the statistical evaluation of the fluorescence intensity shown in the graph on the bottom of FIG. 6. The mean intensity of the coated scaffolds in both NCP and conjugate channel (grey columns) is compared to the mean intensity of the background (black columns). Accordingly, a high fluorescence intensity is determined on the coated scaffolds with a strong contrast to the uncoated background.

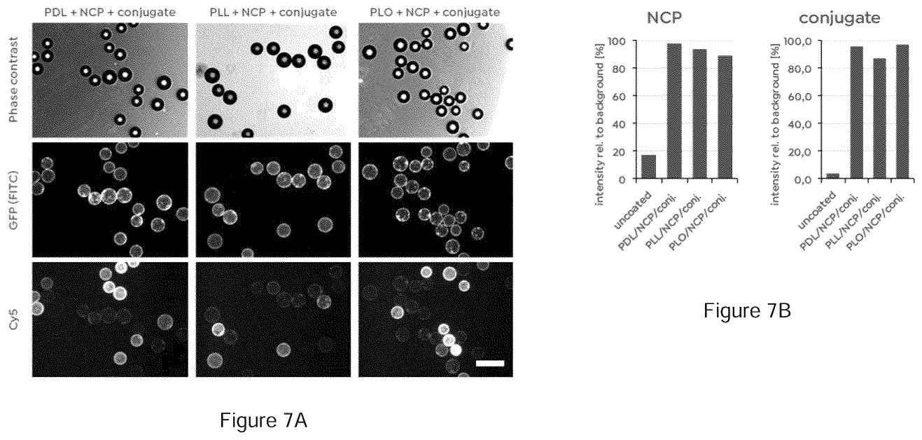

To successfully coat the different materials shown in FIG. 6 with NCP and conjugate, an initial coating with a charge-inducing component (primer) was applied where necessary. Specifically, PDL was used, but other primer materials are also suitable such as poly-L-lysine (PLL) and poly-L-ornithine (PLO). The performance of the three primers PDL, PLL, and PLO was compared in a proof of principle experiment. Polystyrene microcarriers (d=170 μm) have been coated with an initial primer layer followed by a coating with fluorolabeled NCP and conjugate. The fluorescence intensity as a measure for the performance of the different primers is compared in both microscopy images (FIG. 7A) and statistically (FIG. 7B). Similar fluorescence intensities were found in both the images and the statistical evaluation indicating that all three primers perform equally.

To summarize, the coating strategy presented in this invention is highly versatile. Various natural and synthetic scaffold materials can be coated, and it is believed that this portfolio of materials can be further expanded. Additionally, different primer materials have been tested and confirmed to perform equally increasing the flexibility of the approach of the invention even further.

### Modular Characteristics of LbL Coating Enable Optimal Growth of Stem Cells

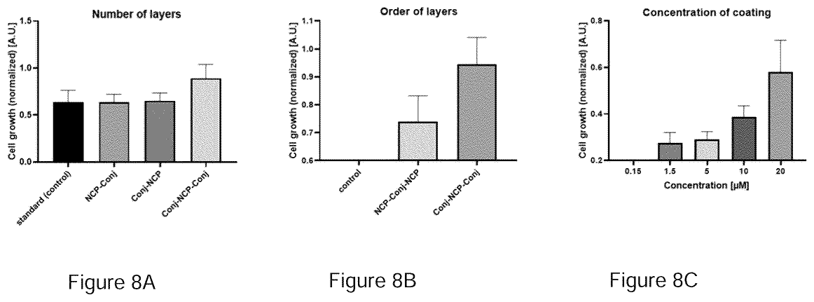

In the given examples the promotion of cell growth was evaluated in respect of the number and order of layers as well as the concentration of the coating material in an LbL approach. The promotion of cell growth of the LbL coating has been evaluated with induced pluripotent stem cells (iPSCs) and mesenchymal stromal cells (MSCs), two anchorage-dependent stem cell types widely used in regenerative medicine and cellular agriculture. To understand the impact of the number of components layered onto each other, MSCs were grown in 2D on standard surfaces (control) or on 2 and 3 layers of the LbL coating (FIG. 8A) and the number of cells was determined at confluence. Both 2-layer coatings performed similar to the standard control, and there was no difference in overall cell growth found when either NCP is deposited ‘outside’ facing the cells or when the conjugate component faces towards cells. However, the 3-layer coating improved the cell growth by ˜0.3-fold, indicating that adjustments in the number of layers can be beneficial for cell growth.

Next, the order of the 3-layer coating was alternated to investigate the impact of the outside layer on cell growth. Although the alternation in 2-layer coatings did not show a difference in cell growth, applying conjugate as last layer improved cell growth by ˜0.25 fold for 3-layer coatings (FIG. 8B). It should be noted that only a few cells grow on the negative control (no coating), so the improved LbL (Conj-NCP-Conj) coating promotes cell growth 8-fold higher than control.

Neither alternation of layers nor the number of layers impacted the short-term growth (<5 days, one passage) of iPSCs in 2D (data not shown). However, modulation of the concentration of the two components showed a strong response of the iPSCs. Analogue to the MSC experiments, iPSCs were seeded onto LbL-coated surfaces in 2D with different coating concentrations. Their cell number was determined after 4 days of growth. Coating concentrations below 1.5 μM showed nearly no cell growth (FIG. 8C). The cell growth improved 0.3-fold when 20 μM instead of 1.5 μM was used.

### Characteristics of LbL Coating and Specific Biofunctional Peptides Enable Culture of Anchorage-Dependent Stem Cells

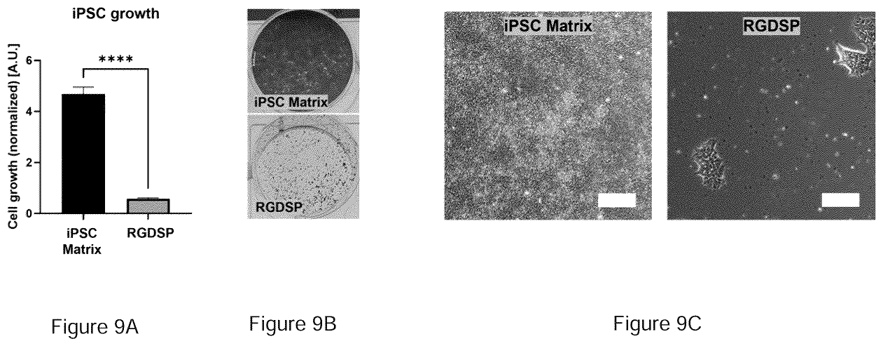

A previous study by Thomas et al. has shown that an LbL coating comprising iterating layers of heparin and conjugate can be applied to cultivate human umbilical vein endothelial cells (HUVECs)¹⁸. In this study, heparin has been coupled covalently using silanization chemistry to provide a starting layer. The applied conjugate contained the biofunctional peptide sequence RGDSP. This is a valuable method for modification of glass surfaces but may not be suitable for polymer-based scaffold materials. Further, this approach may not be biologically relevant for cell types used in cellular agriculture, regenerative medicine, and cell therapy such as MSCs and iPSCs. To test this hypothesis, a polystyrene-based cell culture article was coated with a formulation optimized for iPSC (referred as iPSC MATRIX) comprising of the NPC Dextran sulfate and a conjugate comprising of SEQ ID 6 (see Table 2) in a non-covalent LbL coating approach. The control surfaces have been prepared of heparin and RGDSP-containing conjugate according to the reference. iPSCs were seeded and the number of cells was determined after 4 days. Cells grown on the iPSC MATRIX compared to the RGDSP-coating showed significant (p<0.0001) enhanced cell numbers (FIG. 9A). Analogous to the cell growth measurement, at the time point when the cell reached confluency on the iPSC MATRIX, it was observed that cells on the RGDSP-coating barely grew (FIG. 9B). The remaining cells on the RGDSP-coating had a compromised morphology and the edges of the colonies started to lift off (FIG. 9C). Based on the morphology, it can be assumed that iPSC cannot survive long-term on the RGDSP-coating. In contrast, iPSCs covered the iPSC MATRIX uniformly with expected dense cell morphologies indicating optimal growing conditions.

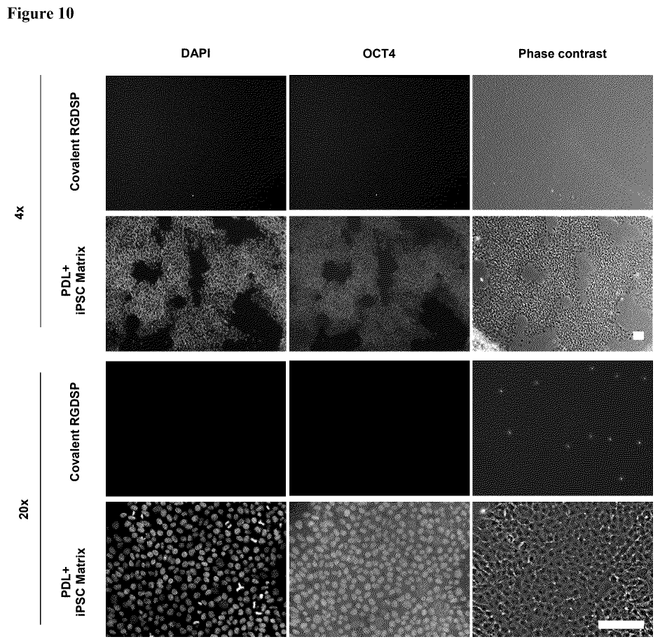

To confirm the superior functionality for stem cells of the presented LbL method over the aforementioned prior art the coating methods were conversely compared. For this heparin and RGDSP were covalently coupled to glass slides analogue to the previous study¹⁸ and compared to glass slides non-covalently coated LbL with PDL, Dextran sulfate and a conjugate containing the biofunctional peptide SEQ ID 6 (see Table 2). The covalently bound RGDSP with Heparin did not lead to any attachment or growth of iPSCs in culture, whereas the LbL coating produced large colonies and a confluent culture after 4-5 days in culture (FIG. 10). This clearly demonstrates that the LbL coating is highly beneficial for stem cell growth in vitro.

In sum, these results show that the LbL is well suited for the culture of stem cells and superior to existing coatings utilizing the combination of conjugate and NCP. The modulatory capacities of the LbL coating allow for improving cell performance and tailoring coatings towards cell type specific needs.

The Incorporation of NCPs Component Stabilizes Growth Factor bFGF

The difference in cell growth on 3 layers with different components facing towards the cells raises the question about the impact of the NCP component on cells. The ability to stabilize growth factors by NCP components alone, e.g. Heparin, has been shown in vitro²². Cell growth, proliferation and differentiation relies on binding of growth factors present in cell culture media. Many cell types, particularly iPSCs, require the presence of high concentrations (<0.05 mg/L) of basic fibroblast growth factor (bFGF or FGF-2) to ensure their maintenance. Those high concentrations are important for cell growth, stemness maintenance and proliferation but are also used since the short half-life of bFGF in cell culture medium reduces their availability rapidly. The half-life of bFGF is also one of the major reasons that iPSC culture generally requires daily media change, a problematic making iPSC culture labor intensive.

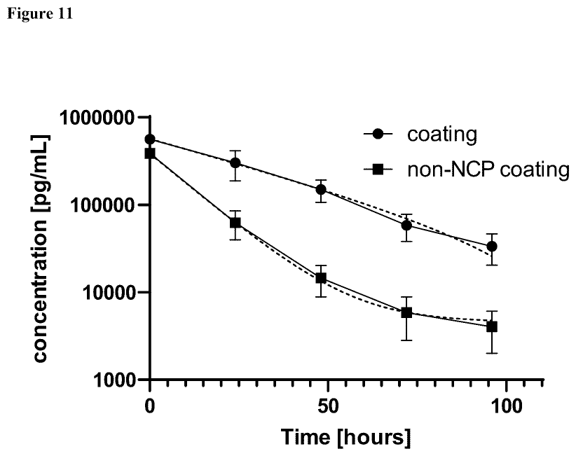

To investigate the impact of an NCP component in the coatings on the half-life of bFGF, the bFGF concentration in mTESR1™ medium over a course of 4 days was measured. Normal mammalian cell culture condition was mimicked in a humidified chamber at 37° C. with 5% CO₂. The concentration of bFGF was measured at each time point for an NCP-containing and a non-NCP-containing coating (FIG. 11). As expected, within 24 hours the bFGF concentration decreases from 0.38 μg/mL to 0.063 μg/mL on the non-NCP containing coating. In contrast, the media incubated on the NCP-coating contained 0.3 μg/mL bFGF after 24 hours, significantly more than the non-NCP coating (p=0.0236, unpaired t-test). The bFGF concentration was also significantly higher at all subsequent time points compared to the non-NCP coating. Using non-linear regression, a one phase decay model (Y=(Y0−Plateau)*exp(−K*X)+Plateau) was fitted to understand the stabilization benefit from the coating containing NCP. The concentration of bFGF on the non-NCP coating after 24 hours is reached on the coating only after 75 hours. The presence of NCP triples the lifetime of bFGF in standard cell culture media and shows that an NCP-containing coating can significantly stabilize growth factors important for mammalian cell culture like bFGF and is therefore an important component in the LbL coating approach of the invention.

### Expansion of Mesenchymal Stromal Cells in 3D Culture

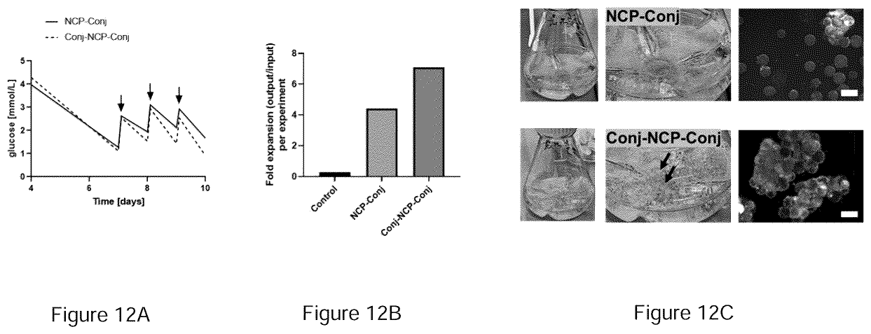

Mesenchymal stromal cells are anchorage-dependent and microcarriers are a frequently chosen 3D scaffold to fulfill this requirement in industrial scale cell culture. Aggregate culture has been attempted but appeared to be most successful for lineage-specific differentiation or to improve functional outcomes^{23 24}. In order to investigate the efficiency of the LbL coating approach, polystyrene microcarriers were coated with 2- and 3-layers and employed for the cultivation of MSC in shaker flasks over 11 days in serum-free media. Surface charge modified polystyrene microcarriers were used as control. The glucose consumption of the cells on microcarriers was monitored over time and used to determine whether media replenishment was necessary (FIG. 12A). At the end of the cultivation period the fold expansion of each condition was determined based on input and output cell numbers (FIG. 12B). Non-coated microcarriers did not support MSC growth in 3D culture. The 2-layer coating allowed for a 5-fold expansion in 3D culture over 11 days. As deduced from the previously shown 2D experiments, the 3-layer coating (Conj-NCP-Conj) resulted in the highest fold expansion, up to ˜7-fold, in 3D culture. The 3-layer LbL coating showed large clumps of cells at the end of the culture period (FIG. 12C) and nearly no beads without cells compared to the 2-layer LbL coating. These experiments show that the LbL coating approach provides relevant results for expansion of stem cells on microcarriers in serum-free media.

### III. CONCLUSION

A chemically defined, animal-component free coating method is provided, that is simple in use, applicable for the culture of cells, with emphasize on primary and stem cells, on plates, and synthetic and natural scaffolds for use in large-scale cell manufacturing, regenerative medicine, and cellular agriculture. The coating material is based on two components, a negatively charged polysaccharide (NCP) and a PEG-peptide-conjugate containing a biofunctional moiety. The conjugate provides the cell interactive construct, which is physically crosslinked by the NCP that further stabilizes mammalian cell culture's growth factors. As the interaction of the individual components is based on charge the invention is universally applicable for all types of scaffolds and allows for transferring cell culture procedures from 2D to 3D, from research scale to industrial scale.

### REFERENCES

1. Jin, Y. L., Hafner, J., Dragieva, G. & Burg, G. Bioreactor Microcarrier Cell Culture System (Bio-MCCS) for large-scale production of autologous melanocytes. Cell Transplant. 13, 809-816 (2004).
2. Bancel, S. & Hu, W. S. Confocal laser scanning microscopy examination of cell distribution in macroporous microcarriers. Biotechnol. Prog. 12, 398-402 (1996).
3. Overstreet, M., Sohrabi, A., Polotsky, A., Hungerford, D. S. & Frondoza, C. G. Collagen microcarrier spinner culture promotes osteoblast proliferation and synthesis of matrix proteins. Vitr. Cell. Dev. Biol. -Anim. 39, 228-234 (2003).
4. William J. Hillegas; David E. Solomon; Gilbert H. Wuttke. MICROCARRIER BEADS HAVING A STYRENE COPOLYMER CORE AND A COVALENTLY LINKED TR METHYLAMINE EXTERIOR. vol. 1 (1999).
5. Kleinman, H. K. et al. Basement Membrane Complexes with Biological Activity. Biochemistry 25, 312-318 (1986).
6. Hughes, C. S., Postovit, L. M. & Lajoie, G. A. Matrigel: a complex protein mixture required for optimal growth of cell culture. Proteomics 10, 1886-1890 (2010).
7. Terranova, V. P., Aumailley, M., Sultan, L. H., Martin, G. R. & Kleinman, H. K. Regulation of cell attachment and cell number by fibronectin and laminin. J. Cell. Physiol. 127, 473-479 (1986).
8. Ruoslahti, E. RGD and other recognition sequences for integrins. Annu. Rev. Cell Dev. Biol. 12, 697-715 (1996).
9. Mouw, J. K., Ou, G. & Weaver, V. M. Extracellular matrix assembly: A multiscale deconstruction. Nature Reviews Molecular Cell Biology vol. 15 771-785 (2014).
10. Afratis, N. et al. Glycosaminoglycans: key players in cancer cell biology and treatment. FEBS J. 279, 1177-1197 (2012).
11. Pickford, C. E. et al. Specific glycosaminoglycans modulate neural specification of mouse embryonic stem cells. Stem Cells 29, 629-640 (2011).
12. Capila, I. & Linhardt, R. J. Heparin—Protein interactions. Angewandte Chemie—International Edition vol. 41 390-412 (2002).
13. Wieduwild, R. et al. Coacervation-Mediated Combinatorial Synthesis of Biomatrices for Stem Cell Culture and Directed Differentiation. Adv. Mater. 30, 1706100 (2018).
14. Wieduwild, R. et al. Noncovalent hydrogel beads as microcarriers for cell culture. Angew. Chemie—Int. Ed. 54, 3962-3966 (2015).
15. Tondera, C. et al. In Vivo Examination of an Injectable Hydrogel System Crosslinked by Peptide-Oligosaccharide Interaction in Immunocompetent Nude Mice. Adv. Funct. Mater. 27, (2017).
16. Zhang, Y., Thomas, A., Wetzel, R., Husman, D. & Wieduwild, R. Non-covalently assembled biomatrix layer. (2017).
17. Thamm, K. et al. A Novel Synthetic, Xeno-Free Biomimetic Surface for Serum-Free Expansion of Human Mesenchymal Stromal Cells. Adv. Biosyst. 4, 2000008 (2020).
18. Thomas, A. K. et al. Layer-by-Layer Assembly of Heparin and Peptide-Polyethylene Glycol Conjugates to Form Hybrid Nanothin Films of Biomatrices. ACS Appl. Mater. Interfaces 10, 14264-14270 (2018).
19. Sukhorukov, G. B. et al. Layer-by-layer self assembly of polyelectrolytes on colloidal particles. Colloids Surfaces A Physicochem. Eng. Asp. 137, 253-266 (1998).
20. Wieduwild, R. et al. Minimal peptide motif for non-covalent peptide-heparin hydrogels (Supporting Information). J. Am. Chem. Soc. 135, 2919-2922 (2013).
21. Mazia, D., Schatten, G. & Sale, W. Adhesion of cells to surfaces coated with polylysine. Applications to electron microscopy. J. Cell Biol. 66, 198-200 (1975).
22. Robert Flaumenhaft, David Moscatelli, and D. B. R. Purification of Heparan Sulfates Secreted by. 111, 1651-1659 (1990).
23. Jossen, V. et al. Mass Production of Mesenchymal Stem Cells—Impact of Bioreactor Design and Flow Conditions on Proliferation and Differentiation. Cells Biomater. Regen. Med. (2014) doi:10.5772/59385.
24. Sart, S., Tsai, A. C., Li, Y. & Ma, T. Three-dimensional aggregates of mesenchymal stem cells: Cellular mechanisms, biological properties, and applications. Tissue Eng.—Part B Rev. 20, 365-380 (2014).
25. Oliveira, M. S. et al., Nanocoatings containing sulfated polysaccharides prepared by layer-by-layer assembly as models to study cell-material interactions. J Mater Chem B, 1, 4406-4418 (2013).

1. A coated article for culturing primary cells and stem cells, said coated article comprising a cell culture scaffold material with a non-covalent coating, wherein said non-covalent coating comprises a multilayer coating comprising a first layer comprising at least one negatively charged polysaccharide (NCP) and a second layer comprising at least one positively charged conjugate.

2. The coated article of claim 1, wherein a surface of the coated article shows a surface charge in a range between −50 mV and 50 mV.

3. The coated article of claim 1, wherein the cell culture scaffold material is selected from synthetic polymers including polystyrene and poly-ethyl(methacrylate) (PEMA) and natural polymers including dextran, starch, alginate, and agarose.

4. The coated article according to claim 1, wherein said cell culture scaffold material comprises a negatively charged surface or a positively charged surface, wherein said multilayer coating comprises a structure (YZ)ₙ or (YZY)ₙ when the cell culture scaffold material comprises the negatively charged surface; or (ZY)ₙ or (ZYZ)ₙ when the cell culture scaffold material comprises the positively charged surface, wherein
Y represents the second layer comprising the at least one positively charged conjugate;
Z represents the first layer comprising the at least one NCP; and
n is an integer between 1 and 100.

5. The coated article according to claim 1, wherein said cell culture scaffold material comprises a neutral surface, and wherein said non-covalent coating comprises
i. a layer comprising a charge-inducing component as a primer layer; and
ii. said multilayer coating comprising a structure (ZY)ₙ or (ZYZ)ₙ, wherein
Y represents the second layer comprising the at least one positively charged conjugate;
Z represents the second layer comprising the at least one NCP; and
n is an integer between 1 and 100.

6. The coated article according to claim 1, wherein said cell culture scaffold material has a three-dimensional structure.

7. The coated article according to claim 1, wherein said NCP poly(sodium-4-styrenesulfonate) (PSS) or a sulfated or phosphorylated oligosaccharide.

8. The coated article according to claim 1, wherein each positively charged conjugate comprises a PEG molecule with a peptide sequence coupled to each end of said PEG molecule, and wherein said peptide sequence consists of a linker peptide (KA)ₙ and a biofunctional peptide, wherein said (KA)ₙ, K is lysine, A is alanine and n is an integer selected from 3, 4, 5, 6, 7, 8, 9, 10, 11, and 12, and wherein said biofunctional peptide is a peptide selected from the group consisting of SEQ ID NOs: 4 to 344.

9. The coated article according to claim 1, wherein the multi-layer coating further comprises a primer layer comprising a charge-inducing component, wherein said charge-inducing component of said primer layer is selected from poly-L-lysine, poly-D-lysine, poly-ornithine, and positively charged polypeptides.

10. The coated article according to claim 1, further comprising primary cells and/or stem cells.

11. A method for preparing a coated article comprising a cell culture scaffold material with a non-covalent coating, comprising the steps of
i. selecting a cell culture scaffold material;
ii. estimating a charge of a surface of the cell culture scaffold material, and
iii. depending on the charge of the surface of the cell culture scaffold, coating said cell culture scaffold with a multilayer coating comprising a structure (YZ)ₙ or (YZY)ₙ when the cell culture scaffold material is estimated to have a negatively charged surface; or (ZY)ₙ or (ZYZ)ₙ when the cell culture scaffold material is estimated to have a positively charged surface, wherein
Y represents a first layer comprising a positively charged conjugate;
Z represents a second layer comprising at least one negatively charged polysaccharide (NCP); and
n is an integer in the range of 1 and 100.

12. The method according to claim 11, wherein said cell culture scaffold material is estimated to have a neutral surface, coating said cell culture scaffold comprises coating said cell surface scaffold with
a layer comprising a charge-inducing component as primer layer; and
the multilayer coating comprising the structure (ZY)ₙ or (ZYZ)ₙ.

13. The method according to claim 11, further comprising the step of incubating the coated article in a culture medium comprising primary cells and/or stem cells.

14. (canceled)

15. (canceled)

16. The coated article of claim 1, wherein said NCP is selected from a group consisting of heparin, heparan sulfate, chondroitin sulfate, dermatan sulfate and keratin sulfate, dextran sulfate, α-cyclodextrin sulfate, β-cyclodextrin sulfate, γ-cyclodextrin sulfate, α-cyclodextrin phosphate, β-cyclodextrin phosphate, and γ-cyclodextrin phosphate.


SEQUENCE LISTING (344 sequences)

SEQ ID NO: 1 - protein, 10 aa, synthetic construct
  REGION 1..10: note = synthetic peptide
KAKAKAKAKA

SEQ ID NO: 2 - protein, 14 aa, synthetic construct
  REGION 1..14: note = synthetic peptide
KAKAKAKAKAKAKA

SEQ ID NO: 3 - protein, 18 aa, synthetic construct
  REGION 1..18: note = synthetic peptide
KAKAKAKAKAKAKAKAKA

SEQ ID NO: 4 - protein, 5 aa, synthetic construct
  REGION 1..5: note = synthetic peptide
RGDSP

SEQ ID NO: 5 - protein, 7 aa, synthetic construct
  REGION 1..7: note = synthetic peptide
FRLVFRY

SEQ ID NO: 6 - protein, 12 aa, synthetic construct
  REGION 1..12: note = synthetic peptide
NGEPRGDTYRAY

SEQ ID NO: 7 - protein, 12 aa, synthetic construct
  REGION 1..12: note = synthetic peptide
NPWHSIYITRFG

SEQ ID NO: 8 - protein, 5 aa, synthetic construct
  REGION 1..5: note = synthetic peptide
PHSRN

SEQ ID NO: 9 - protein, 12 aa, synthetic construct
  REGION 1..12: note = synthetic peptide
PHSRNGGRGDSP

SEQ ID NO: 10 - skipped

SEQ ID NO: 11 - protein, 17 aa, synthetic construct
  REGION 1..17: note = synthetic peptide
CSRNLSEIKLLISRARK

SEQ ID NO: 12 - protein, 15 aa, synthetic construct
  REGION 1..15: note = synthetic peptide
DVDVPDGRGDSLAYG

SEQ ID NO: 13 - protein, 7 aa, synthetic construct
  REGION 1..7: note = synthetic peptide
EIKLLIS

SEQ ID NO: 14 - protein, 14 aa, synthetic construct
  REGION 1..14: note = synthetic peptide
GEFYFDLRLKGDKY

SEQ ID NO: 15 - protein, 6 aa, synthetic construct
  REGION 1..6: note = synthetic peptide
GFPGER

SEQ ID NO: 16 - protein, 13 aa, synthetic construct
  REGION 1..13: note = synthetic peptide
LEPRREVCELNPD

SEQ ID NO: 17 - protein, 9 aa, synthetic construct
  REGION 1..9: note = synthetic peptide
LFSHAVSSN

SEQ ID NO: 18 - protein, 13 aa, synthetic construct
  REGION 1..13: note = synthetic peptide
LGGLPSHYRARNI

SEQ ID NO: 19 - protein, 12 aa, synthetic construct
  REGION 1..12: note = synthetic peptide
NRWHSIYITRFG

SEQ ID NO: 20 - protein, 12 aa, synthetic construct
  REGION 1..12: note = synthetic peptide
PQVTRGDVFTMP

SEQ ID NO: 21 - protein, 21 aa, synthetic construct
  REGION 1..21: note = synthetic peptide
RKIPKASSVPTELSAISMLYL

SEQ ID NO: 22 - protein, 13 aa, synthetic construct
  REGION 1..13: note = synthetic peptide
SIDRVEPYSSTAQ

SEQ ID NO: 23 - protein, 13 aa, synthetic construct
  REGION 1..13: note = synthetic peptide
TFDLLRNSYGVRK

SEQ ID NO: 24 - protein, 17 aa, synthetic construct
  REGION 1..17: note = synthetic peptide
TWSKVGGHLRPGIVQSG

SEQ ID NO: 25 - protein, 12 aa, synthetic construct
  REGION 1..12: note = synthetic peptide
TWYKIAFQRNRK

SEQ ID NO: 26 - protein, 9 aa, synthetic construct
  REGION 1..9: note = synthetic peptide
VAEIDGIEL

SEQ ID NO: 27 - protein, 8 aa, synthetic construct
  REGION 1..8: note = synthetic peptide
VFDNFVLK

SEQ ID NO: 28 - protein, 7 aa, synthetic construct
  REGION 1..7: note = synthetic peptide
QPPRARI

SEQ ID NO: 29 - protein, 16 aa, synthetic construct
  REGION 1..16: note = synthetic peptide
YRSRKYSSWYVALKRK

SEQ ID NO: 30 - protein, 7 aa, synthetic construct
  REGION 1..7: note = Synthetic peptide
ESPLKRQ

SEQ ID NO: 31 - protein, 4 aa, synthetic construct
  REGION 1..4: note = synthetic peptide
RGDS

SEQ ID NO: 32 - protein, 12 aa, synthetic construct
  REGION 1..12: note = synthetic peptide
AAEPLKNIGILF

SEQ ID NO: 33 - protein, 12 aa, synthetic construct
  REGION 1..12: note = synthetic peptide
AASIKVAVSADR

SEQ ID NO: 34 - protein, 12 aa, synthetic construct
  REGION 1..12: note = synthetic peptide
ADNLLFYLGSAK

SEQ ID NO: 35 - protein, 6 aa, synthetic construct
  REGION 1..6: note = synthetic peptide
ADTPPV

SEQ ID NO: 36 - protein, 5 aa, synthetic construct
  REGION 1..5: note = synthetic peptide
AEYLR

SEQ ID NO: 37 - protein, 12 aa, synthetic construct
  REGION 1..12: note = synthetic peptide
AFGVLALWGTRV

SEQ ID NO: 38 - protein, 12 aa, synthetic construct
  REGION 1..12: note = synthetic peptide
AFSTLEGRPSAY

SEQ ID NO: 39 - protein, 12 aa, synthetic construct
  REGION 1..12: note = synthetic peptide
AGQWHRVSVRWG

SEQ ID NO: 40 - protein, 13 aa, synthetic construct
  REGION 1..13: note = synthetic peptide
AGTFALRGDNPQG

SEQ ID NO: 41 - protein, 15 aa, synthetic construct
  REGION 1..15: note = synthetic peptide
AISMLYLDENEKVVL

SEQ ID NO: 42 - protein, 13 aa, synthetic construct
  REGION 1..13: note = synthetic peptide
AKALELRGVQPVS

SEQ ID NO: 43 - protein, 12 aa, synthetic construct
  REGION 1..12: note = synthetic peptide
AKKIKNRLELVV

SEQ ID NO: 44 - protein, 6 aa, synthetic construct
  REGION 1..6: note = synthetic peptide
ALRGDN

SEQ ID NO: 45 - protein, 12 aa, synthetic construct
  REGION 1..12: note = synthetic peptide
ANGQTPIQRYIK

SEQ ID NO: 46 - protein, 16 aa, synthetic construct
  REGION 1..16: note = synthetic peptide
ANRYLAMKEDGRLLAS

SEQ ID NO: 47 - protein, 12 aa, synthetic construct
  REGION 1..12: note = synthetic peptide
ANVTHLLIRANY

SEQ ID NO: 48 - protein, 13 aa, synthetic construct
  REGION 1..13: note = synthetic peptide
APMSGRSPSLVLK

SEQ ID NO: 49 - protein, 13 aa, synthetic construct
  REGION 1..13: note = synthetic peptide
APVKFLGNQVLSY

SEQ ID NO: 50 - protein, 12 aa, synthetic construct
  REGION 1..12: note = synthetic peptide
APVNVTASVQIQ

SEQ ID NO: 51 - protein, 11 aa, synthetic construct
  REGION 1..11: note = synthetic peptide
ASKAIQVFLLA

SEQ ID NO: 52 - protein, 12 aa, synthetic construct
  REGION 1..12: note = synthetic peptide
ASKAIQVFLLAG

SEQ ID NO: 53 - protein, 12 aa, synthetic construct
  REGION 1..12: note = synthetic peptide
ASKAIQVFLLGG

SEQ ID NO: 54 - protein, 9 aa, synthetic construct
  REGION 1..9: note = synthetic peptide
ATETTITIS

SEQ ID NO: 55 - protein, 20 aa, synthetic construct
  REGION 1..20: note = synthetic peptide
  SITE 13: note = misc_feature - Xaa can be any naturally occurring amino acid
ATLQLQEGRLHFXFDLGKGR

SEQ ID NO: 56 - protein, 12 aa, synthetic construct
  REGION 1..12: note = synthetic peptide
AYFSIVKIERVG

SEQ ID NO: 57 - protein, 9 aa, synthetic construct
  REGION 1..9: note = synthetic peptide
CDPGYIGSR

SEQ ID NO: 58 - protein, 19 aa, synthetic construct
  REGION 1..19: note = synthetic peptide
CNYYSNSYSFWLASLNPER

SEQ ID NO: 59 - protein, 17 aa, synthetic construct
  REGION 1..17: note = synthetic peptide
CTDIKGKCTGACDGKQC

SEQ ID NO: 60 - protein, 19 aa, synthetic construct
  REGION 1..19: note = synthetic peptide
DFGTVQLRNGFPFFSYDLG

SEQ ID NO: 61 - protein, 19 aa, synthetic construct
  REGION 1..19: note = synthetic peptide
DFMTLFLAHGRLVFMFNVG

SEQ ID NO: 62 - protein, 4 aa, synthetic construct
  REGION 1..4: note = synthetic peptide
DGEA

SEQ ID NO: 63 - protein, 12 aa, synthetic construct
  REGION 1..12: note = synthetic peptide
DGQWHSVTVSIK

SEQ ID NO: 64 - protein, 10 aa, synthetic construct
  REGION 1..10: note = synthetic peptide
DGRGDSVAYG

SEQ ID NO: 65 - protein, 12 aa, synthetic construct
  REGION 1..12: note = synthetic peptide
DGRWHRVAVIMG

SEQ ID NO: 66 - protein, 6 aa, synthetic construct
  REGION 1..6: note = synthetic peptide
DPETGV

SEQ ID NO: 67 - protein, 8 aa, synthetic construct
  REGION 1..8: note = synthetic peptide
DPGYIGSR

SEQ ID NO: 68 - protein, 5 aa, synthetic construct
  REGION 1..5: note = synthetic peptide
DQNDN

SEQ ID NO: 69 - protein, 18 aa, synthetic construct
  REGION 1..18: note = synthetic peptide
DREGCRRGWVGQCKAWFN

SEQ ID NO: 70 - protein, 10 aa, synthetic construct
  REGION 1..10: note = synthetic peptide
DRVEPYSSTA

SEQ ID NO: 71 - protein, 12 aa, synthetic construct
  REGION 1..12: note = synthetic peptide
DSITKYFQMSLE

SEQ ID NO: 72 - protein, 12 aa, synthetic construct
  REGION 1..12: note = synthetic peptide
DSITKYFQMSLE

SEQ ID NO: 73 - protein, 13 aa, synthetic construct
  REGION 1..13: note = synthetic peptide
DTINNGRDHMILI

SEQ ID NO: 74 - protein, 12 aa, synthetic construct
  REGION 1..12: note = synthetic peptide
DVISLYNFKHIY

SEQ ID NO: 75 - protein, 10 aa, synthetic construct
  REGION 1..10: note = synthetic peptide
DVRRGIKKTD

SEQ ID NO: 76 - protein, 5 aa, synthetic construct
  REGION 1..5: note = synthetic peptide
DWIVA

SEQ ID NO: 77 - protein, 19 aa, synthetic construct
  REGION 1..19: note = synthetic peptide
DYATLQLQEGRLHFMFDLG

SEQ ID NO: 78 - protein, 7 aa, synthetic construct
  REGION 1..7: note = synthetic peptide
EDGIHEL

SEQ ID NO: 79 - protein, 8 aa, synthetic construct
  REGION 1..8: note = synthetic peptide
EILDVPST

SEQ ID NO: 80 - skipped

SEQ ID NO: 81 - protein, 10 aa, synthetic construct
  REGION 1..10: note = synthetic peptide
ERGVVSIKGV

SEQ ID NO: 82 - protein, 7 aa, synthetic construct
  REGION 1..7: note = synthetic peptide
ETITGFG

SEQ ID NO: 83 - protein, 13 aa, synthetic construct
  REGION 1..13: note = synthetic peptide
EVNVTLDLGQVFH

SEQ ID NO: 84 - protein, 15 aa, synthetic construct
  REGION 1..15: note = synthetic peptide
EVYVVAENQQGKSKA

SEQ ID NO: 85 - protein, 11 aa, synthetic construct
  REGION 1..11: note = synthetic peptide
FALWDAIIGEL

SEQ ID NO: 86 - protein, 11 aa, synthetic construct
  REGION 1..11: note = synthetic peptide
FDLHQNMGSVN

SEQ ID NO: 87 - protein, 14 aa, synthetic construct
  REGION 1..14: note = synthetic peptide
FDPELYRSTGHGGH

SEQ ID NO: 88 - protein, 12 aa, synthetic construct
  REGION 1..12: note = synthetic peptide
FFDGSSYAVVRD

SEQ ID NO: 89 - protein, 7 aa, synthetic construct
  REGION 1..7: note = synthetic peptide
FHRRIKA

SEQ ID NO: 90 - protein, 12 aa, synthetic construct
  REGION 1..12: note = synthetic peptide
FQKLLNNLTSIK

SEQ ID NO: 91 - protein, 9 aa, synthetic construct
  REGION 1..9: note = synthetic peptide
FRHRNRKGY

SEQ ID NO: 92 - protein, 12 aa, synthetic construct
  REGION 1..12: note = synthetic peptide
FSTRNESGIILL

SEQ ID NO: 93 - protein, 12 aa, synthetic construct
  REGION 1..12: note = synthetic peptide
FVLYLGSKNAKK

SEQ ID NO: 94 - protein, 6 aa, synthetic construct
  REGION 1..6: note = synthetic peptide
FYFDLR

SEQ ID NO: 95 - protein, 12 aa, synthetic construct
  REGION 1..12: note = synthetic peptide
GAAWKIKGPIYL

SEQ ID NO: 96 - protein, 12 aa, synthetic construct
  REGION 1..12: note = synthetic peptide
GAKGRAGFPGLP

SEQ ID NO: 97 - protein, 6 aa, synthetic construct
  REGION 1..6: note = synthetic peptide
GAPGER

SEQ ID NO: 98 - protein, 12 aa, synthetic construct
  REGION 1..12: note = synthetic peptide
GAPGPKGARGSA

SEQ ID NO: 99 - protein, 6 aa, synthetic construct
  REGION 1..6: note = synthetic peptide
GASGER

SEQ ID NO: 100 - protein, 15 aa, synthetic construct
  REGION 1..15: note = synthetic peptide
GDLGRPGRKGRPGPP

SEQ ID NO: 101 - protein, 13 aa, synthetic construct
  REGION 1..13: note = synthetic peptide
GDRGIKGHRGFSG

SEQ ID NO: 102 - protein, 6 aa, synthetic construct
  REGION 1..6: note = synthetic peptide
  SITE 3: note = MISC_FEATURE - pyrrolysine
GFXGER

SEQ ID NO: 103 - protein, 14 aa, synthetic construct
  REGION 1..14: note = synthetic peptide
GFPGGLNQFGLTTN

SEQ ID NO: 104 - protein, 12 aa, synthetic construct
  REGION 1..12: note = synthetic peptide
GFPGSRGDTGPP

SEQ ID NO: 105 - protein, 12 aa, synthetic construct
  REGION 1..12: note = synthetic peptide
GGFLKYTVSYDI

SEQ ID NO: 106 - protein, 20 aa, synthetic construct
  REGION 1..20: note = synthetic peptide
GGTYSCHFGPLTWVCKPQGG

SEQ ID NO: 107 - protein, 9 aa, synthetic construct
  REGION 1..9: note = synthetic peptide
GGWGRGDSP

SEQ ID NO: 108 - protein, 21 aa, synthetic construct
  REGION 1..21: note = synthetic peptide
GHRGPTGRPGKRGKQGQKGDS

SEQ ID NO: 109 - protein, 6 aa, synthetic construct
  REGION 1..6: note = synthetic peptide
GIIFFL

SEQ ID NO: 110 - protein, 6 aa, synthetic construct
  REGION 1..6: note = synthetic peptide
GKDGES

SEQ ID NO: 111 - protein, 12 aa, synthetic construct
  REGION 1..12: note = synthetic peptide
GKNTGDHFVLYM

SEQ ID NO: 112 - protein, 6 aa, synthetic construct
  REGION 1..6: note = synthetic peptide
GKRGKP

SEQ ID NO: 113 - protein, 10 aa, synthetic construct
  REGION 1..10: note = synthetic peptide
GLDRESYPYY

SEQ ID NO: 114 - protein, 12 aa, synthetic construct
  REGION 1..12: note = synthetic peptide
GLIYYVAHQNQM

SEQ ID NO: 115 - protein, 6 aa, synthetic construct
  REGION 1..6: note = synthetic peptide
GLKGEN

SEQ ID NO: 116 - protein, 12 aa, synthetic construct
  REGION 1..12: note = synthetic peptide
GLLFYMARINHA

SEQ ID NO: 117 - protein, 6 aa, synthetic construct
  REGION 1..6: note = synthetic peptide
GLPGEN

SEQ ID NO: 118 - protein, 6 aa, synthetic construct
  REGION 1..6: note = synthetic peptide
GLPGER

SEQ ID NO: 119 - protein, 6 aa, synthetic construct
  REGION 1..6: note = synthetic peptide
GLSGER

SEQ ID NO: 120 - protein, 6 aa, synthetic construct
  REGION 1..6: note = synthetic peptide
GMPGER

SEQ ID NO: 121 - protein, 13 aa, synthetic construct
  REGION 1..13: note = synthetic peptide
GPAGKDGEAGAQG

SEQ ID NO: 122 - protein, 12 aa, synthetic construct
  REGION 1..12: note = synthetic peptide
GPGVVVVERQYI

SEQ ID NO: 123 - protein, 12 aa, synthetic construct
  REGION 1..12: note = synthetic peptide
GPKGAAGEPGKP

SEQ ID NO: 124 - protein, 12 aa, synthetic construct
  REGION 1..12: note = synthetic peptide
GPLPSYLQFVGI

SEQ ID NO: 125 - protein, 12 aa, synthetic construct
  REGION 1..12: note = synthetic peptide
GPPGDQGPPGIP

SEQ ID NO: 126 - protein, 15 aa, synthetic construct
  REGION 1..15: note = synthetic peptide
GPQGIAGQRGVVGLP

SEQ ID NO: 127 - protein, 13 aa, synthetic construct
  REGION 1..13: note = synthetic peptide
GQLFHVAYILIKF

SEQ ID NO: 128 - protein, 6 aa, synthetic construct
  REGION 1..6: note = synthetic peptide
GQRGER

SEQ ID NO: 129 - protein, 13 aa, synthetic construct
  REGION 1..13: note = synthetic peptide
GQVFHVAYVLIKF

SEQ ID NO: 130 - protein, 6 aa, synthetic construct
  REGION 1..6: note = synthetic peptide
GRRGKT

SEQ ID NO: 131 - protein, 12 aa, synthetic construct
  REGION 1..12: note = synthetic peptide
GSAKFIDFLAIE

SEQ ID NO: 132 - protein, 11 aa, synthetic construct
  REGION 1..11: note = synthetic peptide
GSIKVAVWGGC

SEQ ID NO: 133 - protein, 12 aa, synthetic construct
  REGION 1..12: note = synthetic peptide
GSLSSHLEFVGI

SEQ ID NO: 134 - protein, 13 aa, synthetic construct
  REGION 1..13: note = synthetic peptide
GTNNWWQSPSIQN

SEQ ID NO: 135 - protein, 15 aa, synthetic construct
  REGION 1..15: note = synthetic peptide
GTPGPQGIAGQRDVV

SEQ ID NO: 136 - protein, 15 aa, synthetic construct
  REGION 1..15: note = synthetic peptide
GVKGDKGNPGWPGAP

SEQ ID NO: 137 - protein, 13 aa, synthetic construct
  REGION 1..13: note = synthetic peptide
GVYYQGGTYSKAS

SEQ ID NO: 138 - protein, 9 aa, synthetic construct
  REGION 1..9: note = synthetic peptide
GYIGSRGGC

SEQ ID NO: 139 - skipped

SEQ ID NO: 140 - protein, 7 aa, synthetic construct
  REGION 1..7: note = synthetic peptide
HHHRHSF

SEQ ID NO: 141 - protein, 12 aa, synthetic construct
  REGION 1..12: note = synthetic peptide
HHLGGAKQAGDV

SEQ ID NO: 142 - protein, 12 aa, synthetic construct
  REGION 1..12: note = synthetic peptide
HKIKIVRVKQEG

SEQ ID NO: 143 - protein, 12 aa, synthetic construct
  REGION 1..12: note = synthetic peptide
HKKLKIRSQEKY

SEQ ID NO: 144 - protein, 12 aa, synthetic construct
  REGION 1..12: note = synthetic peptide
HLIMTFKTFRPA

SEQ ID NO: 145 - protein, 38 aa, synthetic construct
  REGION 1..38: note = synthetic peptide
HPIFHRGEFSVADSVSVWVGDCTDIKGKCTGACDGKQC

SEQ ID NO: 146 - protein, 12 aa, synthetic construct
  REGION 1..12: note = synthetic peptide
HQNMGSVNVSVG

SEQ ID NO: 147 - protein, 13 aa, synthetic construct
  REGION 1..13: note = synthetic peptide
HQNQMDYATLQLQ

SEQ ID NO: 148 - protein, 12 aa, synthetic construct
  REGION 1..12: note = synthetic peptide
HRDELLLWARKI

SEQ ID NO: 149 - protein, 6 aa, synthetic construct
  REGION 1..6: note = synthetic peptide
IAFQRN

SEQ ID NO: 150 - protein, 5 aa, synthetic construct
  REGION 1..5: note = synthetic peptide
IDAPS

SEQ ID NO: 151 - protein, 12 aa, synthetic construct
  REGION 1..12: note = synthetic peptide
IENVVTTFAPNR

SEQ ID NO: 152 - protein, 13 aa, synthetic construct
  REGION 1..13: note = synthetic peptide
IISRCQVCMKMRP

SEQ ID NO: 153 - protein, 5 aa, synthetic construct
  REGION 1..5: note = synthetic peptide
IKVAV

SEQ ID NO: 154 - protein, 13 aa, synthetic construct
  REGION 1..13: note = synthetic peptide
IRSLKLTKGTGKP

SEQ ID NO: 155 - protein, 17 aa, synthetic construct
  REGION 1..17: note = synthetic peptide
ISNVFVQRMSQSPEVLD

SEQ ID NO: 156 - protein, 12 aa, synthetic construct
  REGION 1..12: note = synthetic peptide
ISRCQVCMKKRH

SEQ ID NO: 157 - protein, 12 aa, synthetic construct
  REGION 1..12: note = synthetic peptide
ISTVMFKFRTFS

SEQ ID NO: 158 - protein, 12 aa, synthetic construct
  REGION 1..12: note = synthetic peptide
KAFDITYVRLKF

SEQ ID NO: 159 - protein, 13 aa, synthetic construct
  REGION 1..13: note = synthetic peptide
KARSFNVNQLLQD

SEQ ID NO: 160 - protein, 12 aa, synthetic construct
  REGION 1..12: note = synthetic peptide
KDISEKVAVYST

SEQ ID NO: 161 - protein, 12 aa, synthetic construct
  REGION 1..12: note = synthetic peptide
KEAEREVTDLLR

SEQ ID NO: 162 - protein, 6 aa, synthetic construct
  REGION 1..6: note = synthetic peptide
KGHRGF

SEQ ID NO: 163 - protein, 6 aa, synthetic construct
  REGION 1..6: note = synthetic peptide
KGIRGH

SEQ ID NO: 164 - protein, 20 aa, synthetic construct
  REGION 1..20: note = synthetic peptide
KIPKASSVPTELSAISTLYL

SEQ ID NO: 165 - protein, 12 aa, synthetic construct
  REGION 1..12: note = synthetic peptide
KKGSYNNIVVHV

SEQ ID NO: 166 - protein, 16 aa, synthetic construct
  REGION 1..16: note = synthetic peptide
KKQRFRHRNRKGYRSQ

SEQ ID NO: 167 - protein, 6 aa, synthetic construct
  REGION 1..6: note = synthetic peptide
KLDAPT

SEQ ID NO: 168 - protein, 15 aa, synthetic construct
  REGION 1..15: note = synthetic peptide
KLTWQELYQLKYKGI

SEQ ID NO: 169 - protein, 12 aa, synthetic construct
  REGION 1..12: note = synthetic peptide
KMPYVSLELEMR

SEQ ID NO: 170 - protein, 15 aa, synthetic construct
  REGION 1..15: note = synthetic peptide
KNNQKSEPLIGRKKT

SEQ ID NO: 171 - protein, 12 aa, synthetic construct
  REGION 1..12: note = synthetic peptide
KNRLTIELEVRT

SEQ ID NO: 172 - protein, 12 aa, synthetic construct
  REGION 1..12: note = synthetic peptide
KNSFMALYLSKG

SEQ ID NO: 173 - protein, 19 aa, synthetic construct
  REGION 1..19: note = synthetic peptide
KNSFMALYLSKGRLVFALG

SEQ ID NO: 174 - protein, 11 aa, synthetic construct
  REGION 1..11: note = synthetic peptide
KPDVRSYTITG

SEQ ID NO: 175 - protein, 12 aa, synthetic construct
  REGION 1..12: note = synthetic peptide
KPRLQFSLDIQT

SEQ ID NO: 176 - protein, 4 aa, synthetic construct
  REGION 1..4: note = synthetic peptide
KPSS

SEQ ID NO: 177 - protein, 13 aa, synthetic construct
  REGION 1..13: note = synthetic peptide
KQANISIVDIDSN

SEQ ID NO: 178 - protein, 12 aa, synthetic construct
  REGION 1..12: note = synthetic peptide
KQGKALTQRHAK

SEQ ID NO: 179 - protein, 22 aa, synthetic construct
  REGION 1..22: note = synthetic peptide
KQNCLSSRASFRGCVRNLRLSR

SEQ ID NO: 180 - protein, 7 aa, synthetic construct
  REGION 1..7: note = synthetic peptide
KRLDGSV

SEQ ID NO: 181 - protein, 8 aa, synthetic construct
  REGION 1..8: note = synthetic peptide
KRLVTGQR

SEQ ID NO: 182 - protein, 12 aa, synthetic construct
  REGION 1..12: note = synthetic peptide
KRRARDLVHRAE

SEQ ID NO: 183 - protein, 4 aa, synthetic construct
  REGION 1..4: note = synthetic peptide
KRSR

SEQ ID NO: 184 - protein, 12 aa, synthetic construct
  REGION 1..12: note = synthetic peptide
KTWGVYRYFAYD

SEQ ID NO: 185 - protein, 14 aa, synthetic construct
  REGION 1..14: note = synthetic peptide
KVLSRLPYGPGRSK

SEQ ID NO: 186 - protein, 12 aa, synthetic construct
  REGION 1..12: note = synthetic peptide
KVSFLWWVGSGV

SEQ ID NO: 187 - protein, 11 aa, synthetic construct
  REGION 1..11: note = synthetic peptide
KYILRWRPKNS

SEQ ID NO: 188 - protein, 12 aa, synthetic construct
  REGION 1..12: note = synthetic peptide
LAGSCLARFSTM

SEQ ID NO: 189 - protein, 12 aa, synthetic construct
  REGION 1..12: note = synthetic peptide
LAGSCLPVFSTL

SEQ ID NO: 190 - protein, 12 aa, synthetic construct
  REGION 1..12: note = synthetic peptide
LAIKNDNLVYVY

SEQ ID NO: 191 - protein, 10 aa, synthetic construct
  REGION 1..10: note = synthetic peptide
LALERKDHSG

SEQ ID NO: 192 - protein, 12 aa, synthetic construct
  REGION 1..12: note = synthetic peptide
LALFLSNGHFVA

SEQ ID NO: 193 - protein, 5 aa, synthetic construct
  REGION 1..5: note = synthetic peptide
LDVPS

SEQ ID NO: 194 - protein, 12 aa, synthetic construct
  REGION 1..12: note = synthetic peptide
LEAEFHFTHLIM

SEQ ID NO: 195 - protein, 10 aa, synthetic construct
  REGION 1..10: note = synthetic peptide
LEKVAHQLEE

SEQ ID NO: 196 - protein, 6 aa, synthetic construct
  REGION 1..6: note = synthetic peptide
LGTIPG

SEQ ID NO: 197 - protein, 12 aa, synthetic construct
  REGION 1..12: note = synthetic peptide
LHKKGKNSSKPK

SEQ ID NO: 198 - protein, 12 aa, synthetic construct
  REGION 1..12: note = synthetic peptide
LHVFYDFGFSNG

SEQ ID NO: 199 - protein, 20 aa, synthetic construct
  REGION 1..20: note = synthetic peptide
LKKAQINDAKYREISIIYHN

SEQ ID NO: 200 - protein, 12 aa, synthetic construct
  REGION 1..12: note = synthetic peptide
LLEFTSARYIRL

SEQ ID NO: 201 - protein, 10 aa, synthetic construct
  REGION 1..10: note = synthetic peptide
LNRQELFPFG

SEQ ID NO: 202 - protein, 12 aa, synthetic construct
  REGION 1..12: note = synthetic peptide
LNRRYEQARNIS

SEQ ID NO: 203 - protein, 13 aa, synthetic construct
  REGION 1..13: note = synthetic peptide
LPFFNDRPWRRAT

SEQ ID NO: 204 - protein, 12 aa, synthetic construct
  REGION 1..12: note = synthetic peptide
LQQSRIANISME

SEQ ID NO: 205 - protein, 9 aa, synthetic construct
  REGION 1..9: note = synthetic peptide
LQVQLSIRT

SEQ ID NO: 206 - protein, 10 aa, synthetic construct
  REGION 1..10: note = synthetic peptide
LRAHAVDING

SEQ ID NO: 207 - protein, 10 aa, synthetic construct
  REGION 1..10: note = synthetic peptide
LRAHAVDVNG

SEQ ID NO: 208 - protein, 12 aa, synthetic construct
  REGION 1..12: note = synthetic peptide
LSNIDYILIKAS

SEQ ID NO: 209 - protein, 14 aa, synthetic construct
  REGION 1..14: note = synthetic peptide
LTRYKITPRRGPPT

SEQ ID NO: 210 - protein, 12 aa, synthetic construct
  REGION 1..12: note = synthetic peptide
LVFMFNVGHKKL

SEQ ID NO: 211 - protein, 10 aa, synthetic construct
  REGION 1..10: note = synthetic peptide
LVVQAADLQG

SEQ ID NO: 212 - protein, 12 aa, synthetic construct
  REGION 1..12: note = synthetic peptide
LWPLLAVLAAVA

SEQ ID NO: 213 - protein, 13 aa, synthetic construct
  REGION 1..13: note = synthetic peptide
LWVTVRSQQRGLF

SEQ ID NO: 214 - protein, 12 aa, synthetic construct
  REGION 1..12: note = synthetic peptide
MEMQANLLLDRL

SEQ ID NO: 215 - protein, 16 aa, synthetic construct
  REGION 1..16: note = synthetic peptide
MFKKPTPSTLKAGELR

SEQ ID NO: 216 - protein, 12 aa, synthetic construct
  REGION 1..12: note = synthetic peptide
MFRKPIPSTVKA

SEQ ID NO: 217 - protein, 13 aa, synthetic construct
  REGION 1..13: note = synthetic peptide
MILISIGKSQKRM

SEQ ID NO: 218 - protein, 10 aa, synthetic construct
  REGION 1..10: note = synthetic peptide
MKVSATDADD

SEQ ID NO: 219 - protein, 7 aa, synthetic construct
  REGION 1..7: note = synthetic peptide
MNYYSNS

SEQ ID NO: 220 - protein, 12 aa, synthetic construct
  REGION 1..12: note = synthetic peptide
NAPFPKLSWTIQ

SEQ ID NO: 221 - protein, 11 aa, synthetic construct
  REGION 1..11: note = synthetic peptide
NAVGYSVYDIS

SEQ ID NO: 222 - protein, 10 aa, synthetic construct
  REGION 1..10: note = synthetic peptide
NDDGGQFVVT

SEQ ID NO: 223 - protein, 13 aa, synthetic construct
  REGION 1..13: note = synthetic peptide
NEPKVLKSYYYAI

SEQ ID NO: 224 - protein, 12 aa, synthetic construct
  REGION 1..12: note = synthetic peptide
NGQWHKVTAKKI

SEQ ID NO: 225 - skipped

SEQ ID NO: 226 - protein, 9 aa, synthetic construct
  REGION 1..9: note = synthetic peptide
NHGFVVEVT

SEQ ID NO: 227 - protein, 12 aa, synthetic construct
  REGION 1..12: note = synthetic peptide
NIRLRFLRTNTL

SEQ ID NO: 228 - protein, 6 aa, synthetic construct
  REGION 1..6: note = synthetic peptide
NKRAHG

SEQ ID NO: 229 - protein, 12 aa, synthetic construct
  REGION 1..12: note = synthetic peptide
NLLLLLVKANLK

SEQ ID NO: 230 - protein, 7 aa, synthetic construct
  REGION 1..7: note = synthetic peptide
NLLMAAS

SEQ ID NO: 231 - protein, 13 aa, synthetic construct
  REGION 1..13: note = synthetic peptide
NQFGLTTNIRFRG

SEQ ID NO: 232 - protein, 13 aa, synthetic construct
  REGION 1..13: note = synthetic peptide
NQRLASFSNAQQS

SEQ ID NO: 233 - protein, 50 aa, synthetic construct
  REGION 1..50: note = synthetic peptide
NSDSECPLSHDGYCLHDGVCMYIEALDKYACNCVVGYIGERCQYRDLKWW

SEQ ID NO: 234 - protein, 12 aa, synthetic construct
  REGION 1..12: note = synthetic peptide
NVLSLYNFKTTF

SEQ ID NO: 235 - protein, 5 aa, synthetic construct
  REGION 1..5: note = synthetic peptide
PDSGR

SEQ ID NO: 236 - protein, 12 aa, synthetic construct
  REGION 1..12: note = synthetic peptide
PGGMREKGRKAR

SEQ ID NO: 237 - protein, 12 aa, synthetic construct
  REGION 1..12: note = synthetic peptide
PGRWHKVSVRWE

SEQ ID NO: 238 - protein, 5 aa, synthetic construct
  REGION 1..5: note = synthetic peptide
PGVGV

SEQ ID NO: 239 - protein, 9 aa, synthetic construct
  REGION 1..9: note = synthetic peptide
PKGQKGEKG

SEQ ID NO: 240 - protein, 10 aa, synthetic construct
  REGION 1..10: note = synthetic peptide
PLDREAIAKY

SEQ ID NO: 241 - protein, 12 aa, synthetic construct
  REGION 1..12: note = synthetic peptide
PPFLMLLKGSTR

SEQ ID NO: 242 - protein, 26 aa, synthetic construct
  REGION 1..26: note = synthetic peptide
PRARITGYIIKYEKPGSPPREVVPRP

SEQ ID NO: 243 - protein, 14 aa, synthetic construct
  REGION 1..14: note = synthetic peptide
PSKKTKPVKPKKVA

SEQ ID NO: 244 - protein, 10 aa, synthetic construct
  REGION 1..10: note = synthetic peptide
QDPELPDKNM

SEQ ID NO: 245 - protein, 10 aa, synthetic construct
  REGION 1..10: note = synthetic peptide
QGADTPPVGV

SEQ ID NO: 246 - protein, 13 aa, synthetic construct
  REGION 1..13: note = synthetic peptide
QKIAEKFSGTRRG

SEQ ID NO: 247 - protein, 12 aa, synthetic construct
  REGION 1..12: note = synthetic peptide
QQNLGSVNVSTG

SEQ ID NO: 248 - protein, 23 aa, synthetic construct
  REGION 1..23: note = synthetic peptide
QRVEILEGRTECVLSNLRGRTRY

SEQ ID NO: 249 - protein, 12 aa, synthetic construct
  REGION 1..12: note = synthetic peptide
RAYFNGQSFIAS

SEQ ID NO: 250 - protein, 15 aa, synthetic construct
  REGION 1..15: note = synthetic peptide
RDFTKATNIRLRFLR

SEQ ID NO: 251 - protein, 10 aa, synthetic construct
  REGION 1..10: note = synthetic peptide
RDIAEIIKDI

SEQ ID NO: 252 - protein, 12 aa, synthetic construct
  REGION 1..12: note = synthetic peptide
RDQLMTVLANVT

SEQ ID NO: 253 - protein, 19 aa, synthetic construct
  REGION 1..19: note = synthetic peptide
RDSFVALYLSEGHVIFALG

SEQ ID NO: 254 - protein, 12 aa, synthetic construct
  REGION 1..12: note = synthetic peptide
REKYYYAVYDMV

SEQ ID NO: 255 - protein, 12 aa, synthetic construct
  REGION 1..12: note = synthetic peptide
RFDQELRLVSYN

SEQ ID NO: 256 - protein, 9 aa, synthetic construct
  REGION 1..9: note = synthetic peptide
RGQPGRKGL

SEQ ID NO: 257 - protein, 9 aa, synthetic construct
  REGION 1..9: note = synthetic peptide
RGQPGVMGF

SEQ ID NO: 258 - protein, 15 aa, synthetic construct
  REGION 1..15: note = synthetic peptide
RGQPGVPGVPGMKGD

SEQ ID NO: 259 - protein, 9 aa, synthetic construct
  REGION 1..9: note = synthetic peptide
RHVRISRSL

SEQ ID NO: 260 - protein, 20 aa, synthetic construct
  REGION 1..20: note = synthetic peptide
RILSILRHQNLLKELQDLAL

SEQ ID NO: 261 - protein, 17 aa, synthetic construct
  REGION 1..17: note = synthetic peptide
RIQNLLKITNLRIKFVK

SEQ ID NO: 262 - protein, 8 aa, synthetic construct
  REGION 1..8: note = synthetic peptide
RKLKHMRF

SEQ ID NO: 263 - protein, 12 aa, synthetic construct
  REGION 1..12: note = synthetic peptide
RKRLQVQLSIRT

SEQ ID NO: 264 - protein, 21 aa, synthetic construct
  REGION 1..21: note = synthetic peptide
RKVGKASSVPTKLSPISILYK

SEQ ID NO: 265 - protein, 12 aa, synthetic construct
  REGION 1..12: note = synthetic peptide
RLKTRSSHGMIF

SEQ ID NO: 266 - protein, 12 aa, synthetic construct
  REGION 1..12: note = synthetic peptide
RLRGPQRVFDLH

SEQ ID NO: 267 - protein, 13 aa, synthetic construct
  REGION 1..13: note = synthetic peptide
RLVSYNGIIFFLK

SEQ ID NO: 268 - protein, 13 aa, synthetic construct
  REGION 1..13: note = synthetic peptide
RLVSYSGVLFFLK

SEQ ID NO: 269 - protein, 10 aa, synthetic construct
  REGION 1..10: note = synthetic peptide
RNIAEIIKDI

SEQ ID NO: 270 - protein, 12 aa, synthetic construct
  REGION 1..12: note = synthetic peptide
RNRLHLSMLVRP

SEQ ID NO: 271 - protein, 4 aa, synthetic construct
  REGION 1..4: note = synthetic peptide
RPGV

SEQ ID NO: 272 - protein, 13 aa, synthetic construct
  REGION 1..13: note = synthetic peptide
RQVFQVAYIIIKA

SEQ ID NO: 273 - protein, 12 aa, synthetic construct
  REGION 1..12: note = synthetic peptide
RRQTTQAYYAIF

SEQ ID NO: 274 - protein, 25 aa, synthetic construct
  REGION 1..25: note = synthetic peptide
RRWRRWWRRWWRRWRRPVPEYINQS

SEQ ID NO: 275 - protein, 12 aa, synthetic construct
  REGION 1..12: note = synthetic peptide
RRYYYSIKDISV

SEQ ID NO: 276 - protein, 15 aa, synthetic construct
  REGION 1..15: note = synthetic peptide
RSMRLSFRARGYGFR

SEQ ID NO: 277 - protein, 15 aa, synthetic construct
  REGION 1..15: note = synthetic peptide
  SITE 3: note = MISC_FEATURE - norleucine
RTXRLTWRARGYGWR

SEQ ID NO: 278 - protein, 21 aa, synthetic construct
  REGION 1..21: note = synthetic peptide
RTVPKPSSAPTQLNAISTLYF

SEQ ID NO: 279 - protein, 7 aa, synthetic construct
  REGION 1..7: note = synthetic peptide
RYVVLPR

SEQ ID NO: 280 - protein, 13 aa, synthetic construct
  REGION 1..13: note = synthetic peptide
SETTVKYIFRLHE

SEQ ID NO: 281 - protein, 12 aa, synthetic construct
  REGION 1..12: note = synthetic peptide
SFSFRVDRRDTR

SEQ ID NO: 282 - protein, 11 aa, synthetic construct
  REGION 1..11: note = synthetic peptide
SIGFRGDGQTC

SEQ ID NO: 283 - protein, 13 aa, synthetic construct
  REGION 1..13: note = synthetic peptide
SIGLWNYIEREGK

SEQ ID NO: 284 - protein, 12 aa, synthetic construct
  REGION 1..12: note = synthetic peptide
SINNTAVMQRLT

SEQ ID NO: 285 - protein, 7 aa, synthetic construct
  REGION 1..7: note = synthetic peptide
SIYITRF

SEQ ID NO: 286 - protein, 12 aa, synthetic construct
  REGION 1..12: note = synthetic peptide
SLLSQLNNLLDQ

SEQ ID NO: 287 - protein, 12 aa, synthetic construct
  REGION 1..12: note = synthetic peptide
SLVRNRRVITIQ

SEQ ID NO: 288 - protein, 9 aa, synthetic construct
  REGION 1..9: note = synthetic peptide
SPPRRARVT

SEQ ID NO: 289 - protein, 14 aa, synthetic construct
  REGION 1..14: note = synthetic peptide
SPYTFIDSLVLMPY

SEQ ID NO: 290 - protein, 12 aa, synthetic construct
  REGION 1..12: note = synthetic peptide
SQFQESVDNITK

SEQ ID NO: 291 - protein, 13 aa, synthetic construct
  REGION 1..13: note = synthetic peptide
SQRIYQFAKLNYT

SEQ ID NO: 292 - protein, 12 aa, synthetic construct
  REGION 1..12: note = synthetic peptide
SRATAQKVSRRS

SEQ ID NO: 293 - protein, 12 aa, synthetic construct
  REGION 1..12: note = synthetic peptide
SRLRGKNPTKGK

SEQ ID NO: 294 - protein, 12 aa, synthetic construct
  REGION 1..12: note = synthetic peptide
SSFHFDGSGYAM

SEQ ID NO: 295 - protein, 7 aa, synthetic construct
  REGION 1..7: note = synthetic peptide
SVVYGLR

SEQ ID NO: 296 - protein, 12 aa, synthetic construct
  REGION 1..12: note = synthetic peptide
SYWYRIEASRTG

SEQ ID NO: 297 - protein, 12 aa, synthetic construct
  REGION 1..12: note = synthetic peptide
TAGSCLRKFSTM

SEQ ID NO: 298 - protein, 12 aa, synthetic construct
  REGION 1..12: note = synthetic peptide
TAGSCLRRFSTM

SEQ ID NO: 299 - protein, 15 aa, synthetic construct
  REGION 1..15: note = synthetic peptide
TAIPSCPEGTVPLYS

SEQ ID NO: 300 - protein, 15 aa, synthetic construct
  REGION 1..15: note = synthetic peptide
TDIPPCPHGWISLWK

SEQ ID NO: 301 - protein, 13 aa, synthetic construct
  REGION 1..13: note = synthetic peptide
TDIRVTLNRLNTF

SEQ ID NO: 302 - protein, 12 aa, synthetic construct
  REGION 1..12: note = synthetic peptide
TLFLAHGRLVFM

SEQ ID NO: 303 - protein, 20 aa, synthetic construct
  REGION 1..20: note = synthetic peptide
TLFLAHGRLVFMFNVGHKKL

SEQ ID NO: 304 - protein, 12 aa, synthetic construct
  REGION 1..12: note = synthetic peptide
TLGSCLQRFTTM

SEQ ID NO: 305 - protein, 9 aa, synthetic construct
  REGION 1..9: note = synthetic peptide
TLVNSVNSK

SEQ ID NO: 306 - protein, 12 aa, synthetic construct
  REGION 1..12: note = synthetic peptide
TNLRIKFVKLHT

SEQ ID NO: 307 - protein, 12 aa, synthetic construct
  REGION 1..12: note = synthetic peptide
TSAEAYNLLLRT

SEQ ID NO: 308 - protein, 12 aa, synthetic construct
  REGION 1..12: note = synthetic peptide
TSIKIRGTYSER

SEQ ID NO: 309 - protein, 10 aa, synthetic construct
  REGION 1..10: note = synthetic peptide
TTSWSQCSKS

SEQ ID NO: 310 - protein, 12 aa, synthetic construct
  REGION 1..12: note = synthetic peptide
TVFSVDQDNMLE

SEQ ID NO: 311 - protein, 12 aa, synthetic construct
  REGION 1..12: note = synthetic peptide
TWSQKALHHRVP

SEQ ID NO: 312 - protein, 12 aa, synthetic construct
  REGION 1..12: note = synthetic peptide
TWYKIAFQRNRH

SEQ ID NO: 313 - protein, 10 aa, synthetic construct
  REGION 1..10: note = synthetic peptide
TYRIWRDTAN

SEQ ID NO: 314 - protein, 11 aa, synthetic construct
  REGION 1..11: note = synthetic peptide
  SITE 1: note = MISC_FEATURE - seleneocysteine
XEQLERALNSS

SEQ ID NO: 315 - protein, 14 aa, synthetic construct
  REGION 1..14: note = synthetic peptide
VEHDKEFFHPRYHH

SEQ ID NO: 316 - protein, 6 aa, synthetic construct
  REGION 1..6: note = synthetic peptide
VGVAPG

SEQ ID NO: 317 - protein, 12 aa, synthetic construct
  REGION 1..12: note = synthetic peptide
VILQQSAADIAR

SEQ ID NO: 318 - protein, 12 aa, synthetic construct
  REGION 1..12: note = synthetic peptide
VIRDSNVVQLDV

SEQ ID NO: 319 - protein, 10 aa, synthetic construct
  REGION 1..10: note = synthetic peptide
VITVKDINDN

SEQ ID NO: 320 - protein, 12 aa, synthetic construct
  REGION 1..12: note = synthetic peptide
VLIKGGRARKHV

SEQ ID NO: 321 - protein, 12 aa, synthetic construct
  REGION 1..12: note = synthetic peptide
VLLQANDGAGEF

SEQ ID NO: 322 - protein, 12 aa, synthetic construct
  REGION 1..12: note = synthetic peptide
VLVRVERATVFS

SEQ ID NO: 323 - protein, 12 aa, synthetic construct
  REGION 1..12: note = synthetic peptide
VQLRNGFPYFSY

SEQ ID NO: 324 - protein, 12 aa, synthetic construct
  REGION 1..12: note = synthetic peptide
VRWGMQQIQLVV

SEQ ID NO: 325 - protein, 25 aa, synthetic construct
  REGION 1..25: note = synthetic peptide
VSPPRRARVTDATETTITISWRTKT

SEQ ID NO: 326 - protein, 15 aa, synthetic construct
  REGION 1..15: note = synthetic peptide
VVIDASTAIDAPSNL

SEQ ID NO: 327 - protein, 13 aa, synthetic construct
  REGION 1..13: note = synthetic peptide
VVSLYNFEQTFML

SEQ ID NO: 328 - protein, 20 aa, synthetic construct
  REGION 1..20: note = synthetic peptide
WCSRARKQAASIKVAVSADR

SEQ ID NO: 329 - protein, 19 aa, synthetic construct
  REGION 1..19: note = synthetic peptide
WQPPRARITGYIIKYEKPG

SEQ ID NO: 330 - protein, 12 aa, synthetic construct
  REGION 1..12: note = synthetic peptide
WTIQTTVDRGLL

SEQ ID NO: 331 - protein, 20 aa, synthetic construct
  REGION 1..20: note = synthetic peptide
WTPPRAQITGYRLTVGLTRR

SEQ ID NO: 332 - protein, 13 aa, synthetic construct
  REGION 1..13: note = synthetic peptide
WVTVTLDLRQVFQ

SEQ ID NO: 333 - protein, 12 aa, synthetic construct
  REGION 1..12: note = synthetic peptide
YAIFLNKGRLEV

SEQ ID NO: 334 - protein, 9 aa, synthetic construct
  REGION 1..9: note = synthetic peptide
YCWSQYLCY

SEQ ID NO: 335 - protein, 19 aa, synthetic construct
  REGION 1..19: note = synthetic peptide
YEKPGSPPREVVPRPRPGV

SEQ ID NO: 336 - protein, 12 aa, synthetic construct
  REGION 1..12: note = synthetic peptide
YFDGTGFAKAVG

SEQ ID NO: 337 - protein, 5 aa, synthetic construct
  REGION 1..5: note = synthetic peptide
YIGSR

SEQ ID NO: 338 - protein, 10 aa, synthetic construct
  REGION 1..10: note = synthetic peptide
YILHVAVTNY

SEQ ID NO: 339 - protein, 12 aa, synthetic construct
  REGION 1..12: note = synthetic peptide
YIRLRLQRIRTL

SEQ ID NO: 340 - protein, 32 aa, synthetic construct
  REGION 1..32: note = synthetic peptide
YRSRKYSSWYVALKRKHYRSRKYSSWYVALKR

SEQ ID NO: 341 - protein, 16 aa, synthetic construct
  REGION 1..16: note = synthetic peptide
YRVRVTPKEKTGPMKE

SEQ ID NO: 342 - protein, 19 aa, synthetic construct
  REGION 1..19: note = synthetic peptide
YSMKKTTMKIIPFNRLTIG

SEQ ID NO: 343 - protein, 10 aa, synthetic construct
  REGION 1..10: note = synthetic peptide
YTALIIATDN

SEQ ID NO: 344 - protein, 12 aa, synthetic construct
  REGION 1..12: note = Synthetic peptide
YYAISDFAVGGR